(19) United States
(12) Patent Application Publication
(10) Pub. No.: US 2009/0264446 A9
(43) Pub. Date: Oct. 22, 2009

(54) Condensed Pyridines as Kinase Inhibitors

(75) Inventors: Carmen Almansa Rosales, Barcelona (ES); Marina Virgili Bernado, Barcelona (ES); Pedro Manuel Grima Poveda, Oss (NL)

Correspondence Address: ROTHWELL, FIGG, ERNST & MANBECK, P.C., 1425 K STREET, N.W., SUITE 800, WASHINGTON, DC, 20005, US

(73) Assignee: Palau Pharma, S.A., Palau-solita i Plegamans (ES)

(21) Appl. No.: 11/659,271

(22) PCT Filed: Aug. 2, 2005
(86) PCT No.: PCT/EP2005/008371

§ 371 Date: May 18, 2007

(30) Foreign Application Priority Data
Aug. 3, 2004 (ES) 200401971

(51) Int. Cl. A61K 31/4365; C07D 471/00; A61K 31/506; A61P 29/00; C07D 403/14

(52) U.S. Cl. 514/256; 546/114; 544/333; 514/301

(57) ABSTRACT

New compounds of formula (I), wherein the meanings for the various substituents are as disclosed in the description. These compounds are useful as p38 kinase inhibitors.

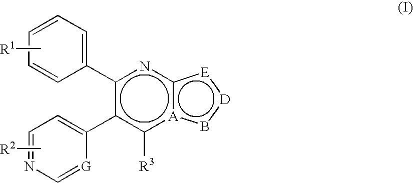

### FIELD OF THE INVENTION

The present invention relates to a new series of heterocyclic compounds, as well as to a process to prepare them, to pharmaceutical compositions comprising these compounds and to their use in therapy.

### BACKGROUND OF THE INVENTION

Kinases are proteins involved in different cellular responses to external signals. In the Nineties, a new family of kinases called MAPK (mitogen-activated protein kinases) was discovered. MAPK activate their substrates by phosphorylation in serine and threonine residues.

MAPK are activated by other kinases in response to a wide range of signals including growth factors, pro-inflammatory cytokines, UV radiation, endotoxins and osmotic stress. Once they are activated, MAPK activate by phosphorylation other kinases or proteins, such as transcription factors, which, ultimately, induce an increase or a decrease in expression of a specific gene or group of genes.

The MAPK family includes kinases such as p38, ERK (extracellular-regulated protein kinase) and JNK (C-Jun N-terminal kinase).

p38 kinase plays a crucial role in cellular response to stress and in the activation pathway in the synthesis of numerous cytokines, especially tumor necrosis factor (TNF-α), interleukin-1 (IL-1), interleukin-6 (IL-6) and interleukin-8 (IL-8).

IL-1 and TNF-α are produced by macrophages and monocytes and are involved in the mediation of immunoregulation processes and other physiopathological conditions. For example, elevated levels of TNF-α are associated with inflammatory and autoimmune diseases and with processes that trigger the degradation of connective and bone tissue such as rheumatoid arthritis, osteoarthritis, diabetes, inflammatory bowel disease and sepsis.

Thus, it is believed that p38 kinase inhibitors can be useful to treat or prevent diseases mediated by cytokines such as IL-1 and TNF-α, such as the ones mentioned above.

On the other hand, it has also been found that p38 inhibitors inhibit other pro-inflammatory proteins such as IL-6, IL-8, interferon-γ and GM-CSF (granulocyte-macrophage colony-stimulating factor). Moreover, in recent studies it has been found that p38 inhibitors do not only block cytokine synthesis but also the cascade of signals that these induce, such as induction of the cyclooxygenase-2 enzyme (COX-2).

Accordingly, it would be desirable to provide novel compounds which are capable of inhibiting the p38 kinase.

### DESCRIPTION OF THE INVENTION

One aspect of the present invention relates to the new compounds of general formula I

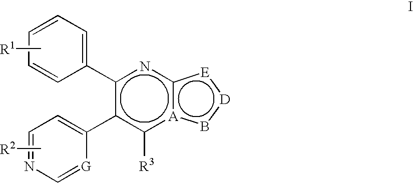

wherein:
A represents C or N;
B, D and E independently represent CR⁴, NR⁵, N, O or S;
with the following provisos:
a) when one of B, D or E represents O or S, the other two cannot represent O or S;
b) when A represents N, none of B, D, E can represent O or S; and
c) when A represents C, B represents CR⁴ and one of D or E represents N or NR⁵, then the other of D or E cannot represent NR⁵ or N;
   G represents N or C;
   R¹ represents one or more substituents selected from H, R^{a}, halogen, —CN, —OH and —OR^{a};
   R² represents one or more substituents selected from H, halogen and C₁₋₆alkyl, and additionally one substituent R² can also represent —OR^{b}, —NO₂, —CN, —COR^{b′}, —CO₂R^{b′}, —CONR^{b′}R^{b′}, —NR^{b′}R^{b′}, —NR^{b′}COR^{b′}, —NR^{b′}CONR^{b′}R^{b′}, —NR^{b′}CO₂R^{b}, —NR^{b′}SO₂R^{b′}, —SR^{b}, —SOR^{b}, —SO₂R^{b}, —SO₂NR^{b′}R^{b′} or C₁₋₆alkyl optionally substituted with one or more substituents R^{c};
   R³ represents:

### H,

C₁₋₆alkyl optionally substituted with one or more substituents selected from R^{c} and R^{d}, or
Cy optionally substituted with one or more substituents selected from R^{c}, R^{d} and C₁₋₆alkyl optionally substituted with one or more substituents selected from R^{c} and R^{d};
each R⁴ independently represents H, R^{e}, halogen, —OR^{e′}, —NO₂, —CN, —COR^{e′}, —CO₂R^{e′}, —CONR^{e′}R^{e′}, —NR^{e′}R^{e′}, —NR^{e′}COR^{e′}, —NR^{e′}CONR^{e′}R^{e′}, —NR^{e′}CO₂R^{e}, —NR^{e′}SO₂R^{e}, —SR^{e′}, —SOR^{e}, —SO₂R^{e} or —SO₂NR^{e′}R^{e′};
R⁵ independently represents H, R^{e}, —COR^{e}, —CONR^{e}R^{e}, —SOR^{e} or —SO₂R^{e};
each R^{a} independently represents C₁₋₆alkyl or haloC₁₋₆alkyl;
each R^{b} independently represents C₁₋₆alkyl or Cy, wherein both groups can be optionally substituted with one or more substituents selected from R^{d} and R^{f};
each R^{b′} independently represents H or R^{b};
each R^{c} independently represents halogen, —OR^{g′}, —NO₂, —CN, —COR^{g′}, —CO₂R^{g′}, —CONR^{g′}R^{g′}, —NR^{g′}R^{g′}, —NR^{g′}COR^{g′}, —NR^{g′}CONR^{g′}R^{g′}, —NR^{g′}CO₂R^{g}, —NR^{g′}SO₂R^{g}, —SR^{g′}, —SOR^{g}, —SO₂R^{g} or —SO₂NR^{g′}R^{g′};
R^{d} represents Cy optionally substituted with one or more substituents R^{f};
each R^{e} independently represents C₁₋₆alkyl optionally substituted with one or more substituents selected from R^{c} and Cy*, or R^{e} represents Cy, wherein any of the groups. Cy or Cy* can be optionally substituted with one or more substituents selected from R^{c} and R^{g};
each R^{e′} independently represents H or R^{e};
each R^{f} independently represents halogen, R^{h}, —OR^{h′}, —NO₂, —CN, —COR^{h′}, CO₂R^{h′}, CONR^{h′}R^{h′}, —NR^{h′}R^{h′}, —NR^{h′}COR^{h′}, —NR^{h′}CONR^{h′}R^{h′}, —NR^{h′}CO₂R^{h}, —NR^{h′}SO₂R^{h}, —SR^{h}, —SOR^{h}, —SO₂R^{h}, or —SO₂NR^{h′}R^{h′};
each R^{g} independently represents R^{d} or C₁₋₆alkyl optionally substituted with one or more substituents selected from R^{d} and R^{f};
each R^{g′} independently represents H or R^{g};
each R^{h} independently represents C₁₋₆alkyl, haloC₁₋₆alkyl or hydroxyC₁₋₆alkyl;
each R^{h} independently represents H or R^{h}; and
Cy or Cy* in the above definitions represent a partially unsaturated, saturated or aromatic 3- to 7-membered monocyclic or 8- to 12-membered bicyclic carbocyclic ring, which optionally contains from 1 to 4 heteroatoms selected from N, S and O, wherein one or more C, N or S atoms can be optionally oxidized forming CO, N⁺O⁻, SO or SO₂, respectively, and wherein said ring or rings can be bonded to the rest of the molecule through a carbon or a nitrogen atom.

The present invention also relates to the salts and solvates of the compounds of formula I.

Some compounds of formula I can have chiral centres that can give rise to various stereoisomers. The present invention relates to each of these stereoisomers and also mixtures thereof.

The compounds of formula I are p38 kinase inhibitors and also inhibit the production of cytokines such as TNF-α.

Thus, another aspect of the invention relates to a compound of general formula I

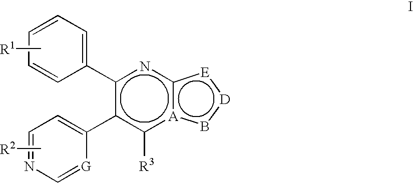

wherein:
A represents C or N;
B, D and E independently represent CR⁴, NR⁵, N, O or S;
with the following provisos:
a) when one of B, D or E represents O or S, the other two cannot represent
O or S;
b) when A represents N, none of B, D, E can represent O or S; and
c) when A represents C, B represents CR⁴ and one of D or E represents N or NR⁵, then the other of D or E cannot represent NR⁵ or N;
   G represents N or C;
   R¹ represents one or more substituents selected from H, R^{a}, halogen, —CN, —OH and —OR^{a};
   R² represents one or more substituents selected from H, halogen and C₁₋₆alkyl, and additionally one substituent R² can also represent —OR^{b′}, —NO₂, —CN, —COR^{b′}, CO₂R^{b′}, —CONR^{b′}R^{b′}, —NR^{b′}R^{b′}, —NR^{b′}COR^{b′}, —NR^{b′}CONR^{b′}R^{b′}, —NR^{b′}CO₂R^{b}, —NR^{b′}SO₂R^{b}, —SR^{b′}, —SOR^{b}, —SO₂R^{b′}, —SO₂NR^{b′}R^{b′} or C₁₋₆alkyl optionally substituted with one or more substituents R^{c};
   R³ represents:

### H,

C₁₋₆alkyl optionally substituted with one or more substituents selected from R^{c} and R^{d} or
Cy optionally substituted with one or more substituents selected from R^{c}, R^{d} and C₁₋₆alkyl optionally substituted with one or more substituents selected from R^{c} and R^{d};
each R⁴ independently represents H, R^{e}, halogen, —OR^{e′}, —NO₂, —CN, —COR^{e′}, —CO₂R^{e′}, —CONR^{e′}R^{e′}, —NR^{e′}R^{e′}, —NR^{e′}COR^{e′}, —NR^{e′}CONR^{e′}R^{e′}, —NR^{e′}CO₂R^{e}, —NR^{e′}SO₂R^{e′}, —SR^{e′}, —SOR^{e}, —SO₂R^{e} or —SO₂NR^{e′}R^{e′};
R⁵ independently represents H, R^{e}, —COR^{e}, —CONR^{e}R^{e}, —SOR^{e} or —SO₂R^{e};
each R^{a} independently represents C₁₋₆alkyl or haloC₁₋₆alkyl;
each R^{b} independently represents C₁₋₆alkyl or Cy, wherein both groups can be optionally substituted with one or more substituents selected from R^{d} and R^{f};
each R^{b′} independently represents H or R^{b};
each R^{c} independently represents halogen, —OR^{g′}, —NO₂, —CN, —COR^{g′}, —CO₂R^{g′}, —CONR^{g′}R^{g′}, NR^{g′}R^{g′}, —NR^{g′}CONR^{g′}R^{g′}, —NR^{g′}CO₂R^{g}, —NR^{g′}SO₂R^{g}, —SR^{g′}, —SOR^{g}, —SO₂R^{g} or —SO₂NR^{g}″R^{g′};
R^{d} represents Cy optionally substituted with one or more substituents R^{f};
each R^{e} independently represents C₁₋₆alkyl optionally substituted with one or more substituents selected from R^{c} and Cy*, or R^{e} represents Cy, wherein any of the groups Cy or Cy* can be optionally substituted with one or more substituents selected from R^{c} and R^{g};
each R^{e}, independently represents H or R^{e};
each R^{f} independently represents halogen, R^{h}, —OR^{h′}, —NO₂, —CN, —COR^{h′}, —CO₂R^{h′}, CONR^{h′}R^{h′}, NR^{h′}R^{h′}, —NR^{h′}COR^{h′}, —NR^{h′}CONR^{h′}R^{h′}, —NR^{h′}CO₂R^{h}, —NR^{h′}SO₂R^{h}, —SR^{h′}, —SOR^{h}, —SO₂R^{h}, or —SO₂NR^{h′}R^{h′};
each R^{g} independently represents R^{d} or C₁₋₆alkyl optionally substituted with one or more substituents selected from R^{d} and R^{f};
each R^{g′} independently represents H or R^{g};
each R^{h} independently represents C₁₋₆alkyl, haloC₁₋₆alkyl or hydroxyC₁₋₆alkyl;
each R^{h′} independently represents H or R^{h}; and
Cy or Cy* in the above definitions represent a partially unsaturated, saturated or aromatic 3- to 7-membered monocyclic or 8- to 12-membered bicyclic carbocyclic ring, which optionally contains from 1 to 4 heteroatoms selected from N, S and O, wherein one or more C, N or S atoms can be optionally oxidized forming CO, N⁺O⁻, SO or SO₂, respectively, and wherein said ring or rings can be bonded to the rest of the molecule through a carbon or a nitrogen atom, for use in therapy.

Another aspect of this invention relates to a pharmaceutical composition which comprises a compound of formula I or a pharmaceutically acceptable salt thereof and one or more pharmaceutically acceptable excipients.

Another aspect of the present invention relates to the use of a compound of formula I or a pharmaceutically acceptable salt thereof for the manufacture of a medicament for the treatment or prevention of diseases mediated by p38.

Another aspect of the present invention relates to the use of a compound of formula I or a pharmaceutically acceptable salt thereof for the manufacture of a medicament for the treatment or prevention of diseases mediated by cytokines.

Another aspect of the present invention relates to the use of a compound of formula I or a pharmaceutically acceptable salt thereof for the manufacture of a medicament for the treatment or prevention of diseases mediated by TNF-α, IL-1, IL-6 and/or IL-8.

Another aspect of the present invention relates to the use of a compound of formula I or a pharmaceutically acceptable salt thereof for the manufacture of a medicament for the treatment or prevention of a disease selected from immune, autoimmune and inflammatory diseases, cardiovascular diseases, infectious diseases, bone resorption disorders, neurodegenerative diseases, proliferative diseases and processes associated with the induction of cyclooxygenase-2.

Another aspect of the present invention relates to the use of a compound of formula I or a pharmaceutically acceptable salt hereof for the treatment or prevention of diseases mediated by p38.

Another aspect of the present invention relates to the use of a compound of formula I or a pharmaceutically acceptable salt thereof for the treatment or prevention of diseases mediated by cytokines.

Another aspect of the present invention relates to the use of a compound of formula I or a pharmaceutically acceptable salt thereof for the treatment or prevention of diseases mediated by TNF-α, IL-1, IL-6 and/or IL-8.

Another aspect of the present invention relates to the use of a compound of formula I or a pharmaceutically acceptable salt thereof for the treatment or prevention of a disease selected from immune, autoimmune and inflammatory diseases, cardiovascular diseases, infectious diseases, bone resorption disorders, neurodegenerative diseases, proliferative diseases and processes associated with the induction of cyclooxygenase-2.

Another aspect of the present invention relates to a method of treating or preventing a disease mediated by p38 in a subject in need thereof, especially a human being, which comprises administering to said subject a therapeutically effective amount of a compound of formula I or a pharmaceutically acceptable salt thereof.

Another aspect of the present invention relates to a method of treating or preventing a disease mediated by cytokines in a subject in need thereof, especially a human being, which comprises administering to said subject a therapeutically effective amount of a compound of formula I or a pharmaceutically acceptable salt thereof.

Another aspect of the present invention relates to a method of treating or preventing a disease mediated by TNF-α, IL-1, IL-6 and/or IL-8 in a subject in need thereof, especially a human being, which comprises administering to said subject a therapeutically effective amount of a compound of formula I or a pharmaceutically acceptable salt thereof.

Another aspect of the present invention relates to a method of treating or preventing a disease selected from immune, autoimmune and inflammatory diseases, cardiovascular diseases, infectious diseases, bone resorption disorders, neurodegenerative diseases, proliferative diseases and processes associated with the induction of cyclooxygenase-2 in a subject in need thereof, especially a human being, which comprises administering to said subject a therapeutically effective amount of a compound of formula I or a pharmaceutically acceptable salt thereof.

Another aspect of the present invention relates to a process for the preparation of a compound of formula I, which comprises:

(a) when in a compound of formula I A represents C, reacting a ketone of formula IV

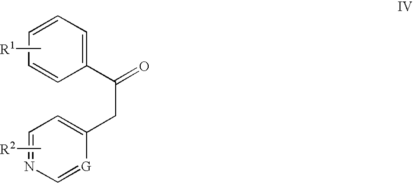

wherein G, R¹ and R² have the meaning described in general formula I, with a heterocyclic amine of formula III and an aldehyde of formula II

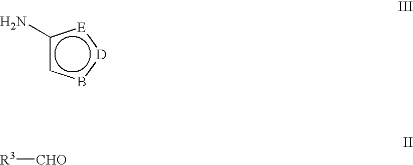

wherein B, D, E and R³ have the meaning described in general formula I; or
(b) when in a compound of formula I A represents N and R³ represents a group identical to the phenyl substituted with R¹ placed on the adjacent position to the N atom of the 6-membered ring of the central bicyclic moiety, reacting a compound of formula XXII

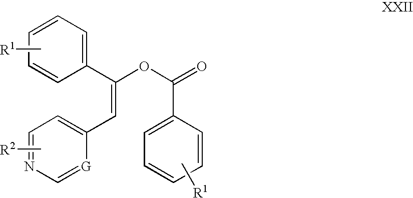

wherein G, R¹ and R² have the meaning described in general formula I, with a heterocyclic amine of formula XXIII

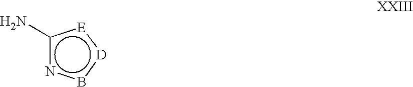

wherein B, D and E have the meaning described in general formula I; or
(c) converting, in one or a plurality of steps, a compound of formula I into another compound of formula I; and
(d) if desired, after any of the above steps a, b or c, reacting a compound of formula I with a base or an acid to give the corresponding salt.

Another aspect of the present invention relates to a process for the preparation of a compound of formula

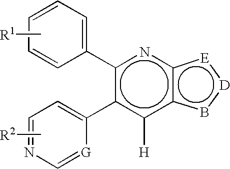

which comprises reacting a propenone of formula

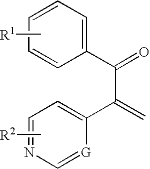

wherein G, R¹ and R² have the previously indicated meanings, with a heterocyclic amine of formula

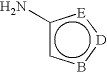

wherein B, D and E independently represent CR⁴, NR⁵, N, O or S; with the proviso that when one of B, D or E represents O or S, the other two cannot represent O or S; and R⁴ and R⁵ have the previously indicated meanings.

In the definitions of the present invention, the term C₁₋₆alkyl, as a group or part of a group, means a straight or branched alkyl chain which contains from 1 to 6 carbon atoms. Examples include among others the groups methyl, ethyl, propyl, isopropyl, butyl, isobutyl, sec-butyl, tert-butyl, pentyl, isopentyl, neopentyl and hexyl.

A haloC₁₋₆alkyl group means a group resulting from the replacement of one or more hydrogen atoms from a C₁₋₆alkyl group with one or more halogen atoms (i.e. fluoro, chloro, bromo or iodo), which can be the same or different. Examples include among others the groups trifluoromethyl, fluoromethyl, 1-chloroethyl, 2-chloroethyl, 1-fluoroethyl, 2-fluoroethyl, 2-bromoethyl, 2-iodoethyl, 2,2,2-trifluoroethyl, pentafluoroethyl, 3-fluoropropyl, 3-chloropropyl, 2,2,3,3-tetrafluoropropyl, 2,2,3,3,3-pentafluoropropyl, heptafluoropropyl, 4-fluorobutyl, nonafluorobutyl, 5-fluoropentyl and 6-fluorohexyl.

A hydroxyC₁₋₆alkyl group means a group resulting from the replacement of one or more hydrogen atoms from a C₁₋₆alkyl group with one or more —OH groups. Examples include among others the groups hydroxymethyl, 1-hydroxyethyl, 2-hydroxyethyl, 1,2-dihydroxyethyl, 3-hydroxypropyl, 4-hydroxybutyl, 5-hydroxypentyl and 6-hydroxyhexyl.

A halogen radical means fluoro, chloro, bromo or iodo.

The term Cy or Cy*, as a group or part of a group, relates to a 3- to 7-membered monocyclic or 8- to 12-membered bicyclic carbocyclic group which can be partially unsaturated, saturated or aromatic, which optionally contains from 1 to 4 heteratoms selected from N, S and O and wherein said ring or rings can be bonded to the rest of the molecule through a carbon or nitrogen atom. When the Cy or Cy* group is saturated or partially unsaturated, one or more C or S atoms can be optionally oxidized, forming a CO, SO or SO₂ group. When the Cy or Cy* group is aromatic, one or more N atoms can be optionally oxidized, forming a N⁺O⁻ group. The Cy or Cy* ring can be substituted as disclosed in the definition of general formula I; if substituted, the substituents can be the same or different and can be placed on any available position. The Cy or Cy* group can be bonded to the rest of the molecule through any available carbon or nitrogen atom. Preferably, the group Cy or Cy* is a 3- to 7-membered monocyclic ring. Examples of Cy or Cy* groups include among others cyclopropyl, cyclobutyl, cyclopentyl, cyclohexyl, cycloheptyl, aziridinyl, oxiranyl, oxetanyl, imidazolidinyl, isothiazolidinyl, isoxazolidinyl, oxazolidinyl, pyrazolidinyl, pyrrolidinyl, thiazolidinyl, dioxanyl, morpholinyl, piperazinyl, piperidinyl, pyranyl, tetrahydropyranyl, azepinyl, oxazinyl, oxazolinyl, pyrrolinyl, thiazolinyl, pyrazolinyl, imidazolinyl, isoxazolinyl, isothiazolinyl, phenyl, naphthyl, 1,2,4-oxadiazolyl, 1,2,4-thiadiazolyl, 1,3,4-oxadiazolyl, 1,3,4-thiadiazolyl, furyl, imidazolyl, isoxazolyl, isothiazolyl, oxazolyl, pyrazolyl, pyrrolyl, thiazolyl, thienyl, 1,2,3-triazolyl, 1,2,4-triazolyl, pyrazinyl, pyridazinyl, pyridinyl, pyrimidinyl, benzimidazolyl, benzofuranyl, isobenzofuranyl, benzothiazolyl, benzothiophenyl, isobenzotiophenyl, imidazopyrazinyl, imidazopyridazinyl, imidazopyridinyl, imidazopyrimidinyl, indazolyl, indolyl, isoindolyl, isoquinolinyl, tetrahydroisoquinolinyl, naphthyridinyl, pyrazolopyrazinyl, pyrazolopyridinyl, pyrazolopyrimidinyl, purinyl, quinazolinyl, quinolinyl, quinoxalinyl, cyclobutanonyl, cyclopentanonyl, cyclohexanonyl, cycloheptanonyl, 2-oxo-pyrrolidinyl, 2-oxo-piperidinyl, 4-oxo-piperidinyl, 2(1H)-pyridonyl, 2(1H)-pyrazinonyl, 2(1H)-pyrimidinonyl, 2(1H)-pyridazinonyl and phthalimidyl.

The term heteroaryl means an aromatic 5- or 6-membered monocyclic or 8- to 12-membered bicyclic ring which contains from 1 to 4 heteroatoms selected from N, S and O, N atoms in the ring can be optionally oxidized forming N⁺O⁻. The heteroaryl group can be linked to the rest of the molecule through any available carbon or nitrogen atom. The heteroaryl group can be optionally substituted as disclosed whenever this term is used; if substituted, the substituents can be the same or different and can be placed on any available position in the ring. Preferably, the heteroaryl group is a 5- or 6-membered monocyclic ring. Examples of heteroaryl groups include among others 1,2,4-oxadiazolyl, 1,2,4-thiadiazolyl, 1,3,4-oxadiazolyl, 1,3,4-thiadiazolyl, furyl, imidazolyl, isoxazolyl, isothiazolyl, oxazolyl, pyrazolyl, pyrrolyl, thiazolyl, thienyl, 1,2,3-triazolyl, 1,2,4-triazolyl, pyrazinyl, pyridazinyl, pyridinyl, pyrimidinyl, benzimidazolyl, benzofuranyl, benzothiazolyl, benzothiophenyl, imidazopyrazinyl, imidazopyridazinyl, imidazopyridinyl, imidazopyrimidinyl, indazolyl, indolyl, isoindolyl, isoquinolinyl, naphthiridinyl, pyrazolopyrazinyl, pyrazolopyridinyl, pyrazolopyrimidinyl, purinyl, quinazolinyl, quinolinyl and quinoxalinyl.

In the definitions of heteroaryl, Cy and Cy*, when the specified examples refer to a bicyclic ring in general terms, all possible dispositions of the atoms are included. Thus for example, the term pyrazolopyridinyl can include groups such as 1H-pyrazolo[3,4-b]pyridinyl, pyrazolo[1,5-a]pyridinyl, 1H-pyrazolo[3,4-c]pyridinyl, 1H-pyrazolo[4,3-c]pyridinyl and 1H-pyrazolo[4,3-b]pyridinyl; the term imidazopyrazinyl can include groups such as 1H-imidazo[4,5-b]pyrazinyl, imidazo[1,2-a]pyrazinyl and imidazo[1,5-a]pyrazinyl and the term pyrazolopyrimidinyl can include groups such as 1H-pyrazolo[3,4-d]pyrimidinyl, 1H-pyrazolo[4,3-d]pyrimidinyl, pyrazolo[1,5-a]pyrimidinyl and pyrazolo[1,5-c]pyrimidinyl.

The expression “optionally substituted with one or more” means that a group can be substituted with one or more, preferably with 1, 2, 3 or 4 substituents, provided that this group has 1, 2, 3 or 4 positions susceptible of being substituted.

In the definition of a compound of formula I, the central bicyclic ring

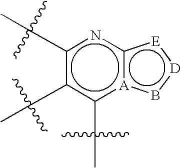

represents an aromatic ring.

In a compound of formula I, R¹ represents one or more, preferably one or two, groups independently selected from H, R^{a}, halogen, —CN, —OH and —OR^{a}. The group or groups R¹ can be placed upon any available position of the phenyl ring and when there is more than one R¹ group, they can be the same or different.

In a compound of formula I, R² represents one or more, preferably one or two, groups independently selected from H, halogen and C₁₋₆alkyl, and additionally one substituent R² can also represent —OR^{b′}, —NO₂, —CN, —COR^{b′}, —CO₂R^{b′}, —CONR^{b′}R^{b′}, —NR^{b′}R^{b′}, —NR^{b′}COR^{b′}, —NR^{b′}CONR^{b′}R^{b′}, —NR^{b′}CO₂R^{b}, —NR^{b′}SO₂R^{b}, SR^{b′}, SOR^{b}, SO₂R^{b}, —SO₂NR^{b′}R^{b′} or C₁₋₆alkyl optionally substituted with one or more substituents R^{c}. The group or groups R² can be placed upon any available carbon atom of the pyridine or pyrimidine ring, including G when G represents C.

The invention thus relates to the compounds of formula I as defined here above.

In another embodiment, the invention relates to the compounds of formula I wherein R¹ represents one or more substituents selected from H, R^{a}, halogen and —OR^{a}.

In another embodiment, the invention relates to the compounds of formula I wherein R¹ represents one or more substituents selected from H, halogen, haloC₁₋₆alkyl and —OR^{a} wherein R^{a} represents C₁₋₆alkyl.

In another embodiment, the invention relates to the compounds of formula I wherein R¹ represents one or two substituents selected from halogen, haloC₁₋₆alkyl and —OR^{a} wherein R^{a} represents C₁₋₆alkyl.

In another embodiment, the invention relates to the compounds of formula I wherein R¹ represents one or more substituents selected from H, halogen and haloC₁₋₆alkyl.

In another embodiment, the invention relates to the compounds of formula I wherein R¹ represents one or more substituents selected from halogen (preferably fluoro) and haloC₁₋₆alkyl (preferably CF₃).

In a further embodiment, the invention relates to the compounds of formula I wherein R¹ represents one or more halogen atoms.

In a further embodiment, the invention relates to the compounds of formula I wherein R² represents one substituent selected from H, halogen, C₁₋₆alkyl, —OR^{b}, —NR^{b′}COR^{b′} and —NR^{b′}R^{b′}.

In a further embodiment, the invention relates to the compounds of formula I wherein R² represents one substituent selected from H, halogen, C₁₋₆alkyl, —OR^{b′}. and —NR^{b′}R^{b′}.

In a further embodiment, the invention relates to the compounds of formula I wherein R² represents one substituent selected from H and —NR^{b′}R^{b′}.

In a further embodiment, the invention relates to the compounds of formula I wherein G represents C and R² represents H.

In a further embodiment, the invention relates to the compounds of formula I wherein G represents N and R² represents —NR^{b′}R^{b′} and is placed on the 2-position of the pyrimidine ring.

In a further embodiment, the invention relates to the compounds of formula I wherein G represents N, R² represents —NHR^{b} and is placed on the 2-position of the pyrimidine ring, and R^{b} represents C₁₋₆alkyl substituted with one substituent selected from Cy and —OR^{h}.

In a further embodiment, the invention relates to the compounds of formula I wherein R³ represents H or Cy optionally substituted with one or more substituents selected from R^{c}, R^{d} and C₁₋₆alkyl optionally substituted with one or more substituents selected from R^{c} and R^{d}.

In a further embodiment, the invention relates to the compounds of formula I wherein R³ represents H, heteroaryl or phenyl, wherein heteroaryl and phenyl can be optionally substituted with one or more substituents selected from R^{c}, R^{d} and C₁₋₆alkyl optionally substituted with one or more substituents selected from R^{c} and R^{d}.

In a further embodiment, the invention relates to the compounds of formula I wherein R³ represents H, heteroaryl or phenyl, wherein heteroaryl and phenyl can be optionally substituted with one or more halogen atoms.

In a further embodiment, the invention relates to the compounds of formula I wherein R³ represents H or phenyl optionally substituted with one or more halogen atoms.

In a further embodiment, the invention relates to the compounds of formula I wherein R³ represents H.

In a further embodiment, the invention relates to the compounds of formula I wherein R³ represents Cy optionally substituted with one or more substituents selected from R^{c}, R^{d} and C₁₋₁₆alkyl optionally substituted with one or more substituents selected from R^{c} and R^{d}.

In a further embodiment, the invention relates to the compounds of formula I wherein R³ represents heteroaryl or phenyl, wherein heteroaryl and phenyl can be optionally substituted with one or more substituents selected from R^{c}, R^{d} and C₁₋₆alkyl optionally substituted with one or more substituents selected from R^{c} and R^{d}.

In a further embodiment, the invention relates to the compounds of formula I wherein R³ represents heteroaryl or phenyl, wherein heteroaryl and phenyl can be optionally substituted with one or more halogen atoms.

In a further embodiment, the invention relates to the compounds of formula I wherein R³ represents phenyl optionally substituted with one or more halogen atoms.

In a further embodiment, the invention relates to the compounds of formula I wherein G represents C, R² represents H and R³ represents heteroaryl or phenyl, wherein heteroaryl and phenyl can be optionally substituted with one or more substituents selected from R^{c}, R^{d} and C₁₋₆alkyl optionally substituted with one or more substituents selected from R^{c} and R^{d}.

In a further embodiment, the invention relates to the compounds of formula I wherein G represents C, R² represents H and R³ represents heteroaryl or phenyl, wherein heteroaryl and phenyl can be optionally substituted with one or more halogen atoms.

In a further embodiment, the invention relates to the compounds of formula I wherein G represents C, R² represents H and R³ represents phenyl optionally substituted with one or more halogen atoms.

In a further embodiment, the invention relates to the compounds of formula I wherein G represents N, R² represents —NR^{b′}R^{b′} and is placed on the 2-position of the pyrimidine ring, and R³ represents H.

In a further embodiment, the invention relates to the compounds of formula I wherein G represents N, R² represents —NHR^{b} and is placed on the 2-position of the pyrimidine ring, R^{b} represents C₁₋₆alkyl substituted with one substituent selected from Cy and —OR^{h′}, and R³ represents H.

In a further embodiment, the invention relates to the compounds of formula I wherein R⁴ independently represents H, R^{e}, —COR^{e′}, —CO₂R^{e′}, —CONR^{e′}R^{e′} or —NR^{e′}R^{e′}.

In a further embodiment, the invention relates to the compounds of formula I wherein R⁴ independently represents H, —COR^{e}, —CONR^{e′}R^{e′} or C₁₋₆alkyl optionally substituted with one or more substituents selected from R^{c}.

In a further embodiment, the invention relates to the compounds of formula I wherein R⁴ independently represents H, —COR^{e′}, —CONR^{e′}R^{e′}, C₁₋₆alkyl, hydroxyC₁₋₆alkyl or —CH₂NR^{g′}R^{g′}.

In a further embodiment, the invention relates to the compounds of formula I wherein R⁵ represents H or R^{e}.

In a further embodiment, the invention relates to the compounds of formula I wherein R⁵ represents H or C₁₋₆alkyl.

In a further embodiment, the invention relates to the compounds of formula I wherein R⁵ represents C₁₋₆alkyl.

In a further embodiment, the invention relates to the compounds of formula I wherein A represents C.

In a further embodiment, the invention relates to the compounds of formula I wherein A represents N.

In a further embodiment, the invention relates to the compounds of formula I wherein

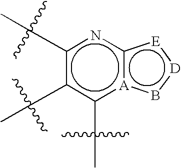

represents a group selected from (a)-(h)

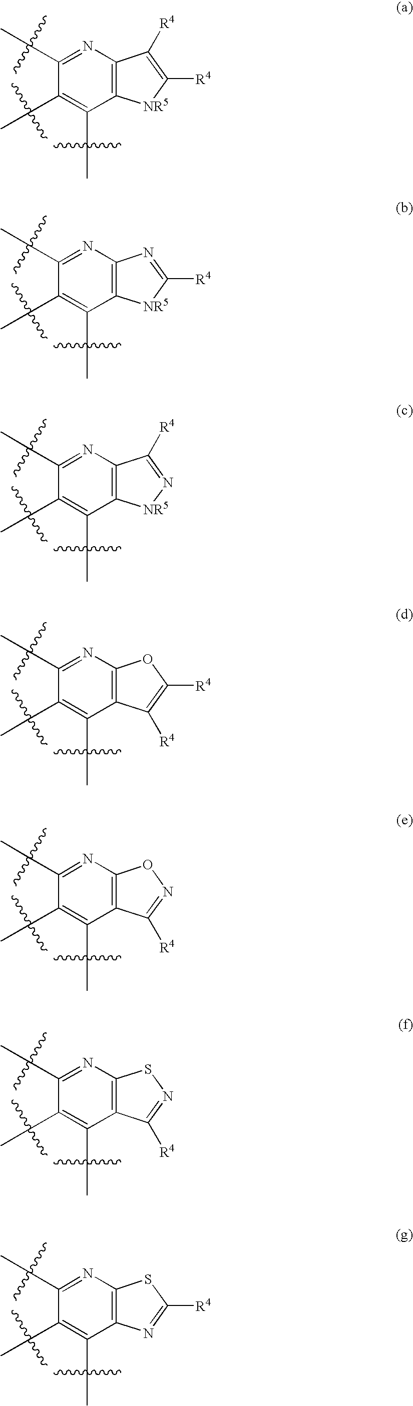

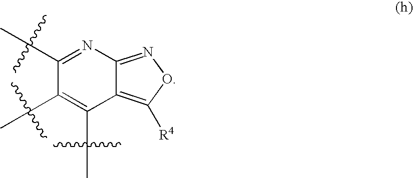

In a further embodiment, the invention relates to the compounds of formula I wherein

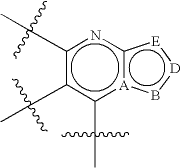

represents a group selected from (a)-(d)

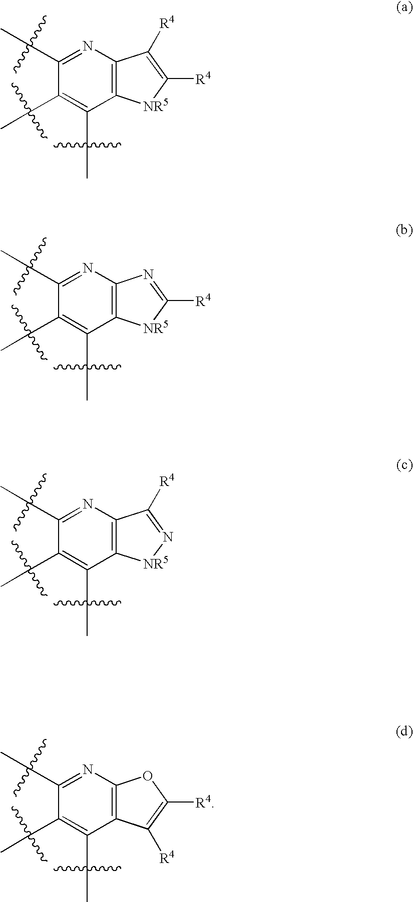

In a further embodiment, the invention relates to the compounds of formula I wherein

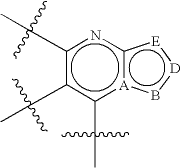

represents a group selected from (a)-(c)

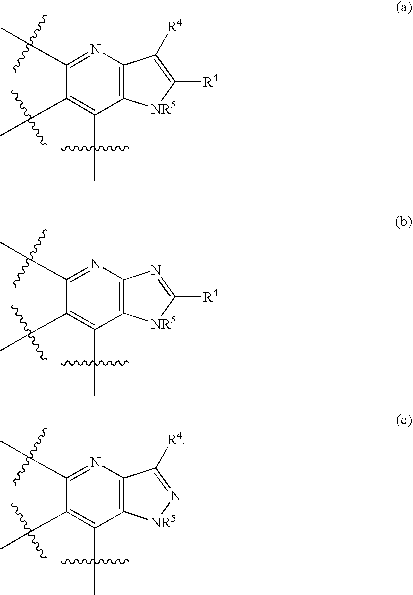

In a further embodiment, the invention relates to the compounds of formula I wherein A represents C; B and D represent CR⁴ and E represents O.

In a further embodiment, the invention relates to the compounds of formula I wherein A represents C; D and E represent CR⁴ and B represents NR⁵.

In a further embodiment, the invention relates to the compounds of formula I wherein A represents C; D represents CR⁴ and one of B and E represents N and the other of B and E represents NR⁵.

In a further embodiment, the invention relates to the compounds of formula I wherein A represents C; D represents CR⁴, E represents N and B represents NR⁵.

In a further embodiment, the invention relates to the compounds of formula I wherein A represents C; E represents CR⁴, D represents N and B represents NR⁵.

In all the above embodiments, all groups for which no specific definition is herein given have the meaning previously indicated in relation to a compound of formula I.

Furthermore, the present invention covers all possible combinations of particular and preferred groups described hereinabove.

In a further embodiment, the invention relates to compounds according to formula I above which provide more than 50% inhibition of p38 activity at 10 μM, more preferably at 1 μM and still more preferably at 0.1 μM, in a p38 assay such as the one described in Example 57.

The compounds of the present invention contain one or more basic nitrogens and may, therefore, form salts with organic or inorganic acids. Examples of these salts include: salts with inorganic acids such as hydrochloric acid, hydrobromic acid, hydroiodic acid, nitric acid, perchloric acid, sulfuric acid or phosphoric acid; and salts with organic acids such as methanesulfonic acid, trifluoromethanesulfonic acid, ethanesulfonic acid, benzenesulfonic acid, p-toluenesulfonic acid, fumaric acid, oxalic acid, acetic acid, maleic acid, ascorbic acid, citric acid, lactic acid, tartaric acid, malonic acid, glycolic acid, succinic acid and propionic acid, among others. Some of the compounds of the present invention may contain one or more acidic protons and, therefore, they may also form salts with bases. Examples of these salts include: salts with inorganic cations such as sodium, potassium, calcium, magnesium, lithium, aluminium, zinc, etc; and salts formed with pharmaceutically acceptable amines such as ammonia, alkylamines, hydroxylalkylamines, lysine, arginine, N-methylglucamine, procaine and the like.

There is no limitation on the type of salt that can be used, provided that these are pharmaceutically acceptable when they are used for therapeutic purposes. The term pharmaceutically acceptable salt represents those salts which are, according to medical judgement, suitable for use in contact with the tissues of humans and other mammals without undue toxicity, irritation, allergic response and the like. Pharmaceutically acceptable salts are well known in the art.

The salts of a compound of formula I can be obtained during the final isolation and purification of the compounds of the invention or can be prepared by treating a compound of formula I with a sufficient amount of the desired acid or base to give the salt in the conventional manner. The salts of the compounds of formula I can be converted into other salts of the compounds of formula I by ion exchange using ionic exchange resins.

The compounds of formula I and their salts may differ in some physical properties but they are equivalent for the purposes of the present invention. All salts of the compounds of formula I are included within the scope of the invention.

The compounds of the present invention may form complexes with solvents in which they are reacted or from which they are precipitated or crystallized. These complexes are known as solvates. As used herein, the term solvate refers to a complex of variable stoichiometry formed by a solute (a compound of formula I or a salt thereof) and a solvent. Examples of solvents include pharmaceutically acceptable solvents such as water, ethanol and the like. A complex with water is known as a hydrate. Solvates of compounds of the invention (or salts thereof), including hydrates, are included within the scope of the invention.

Some of the compounds of the present invention may exist as several diastereoisomers and/or several optical isomers. Diastereoisomers can be separated by conventional techniques such as chromatography or fractional crystallization. Optical isomers can be resolved by conventional techniques of optical resolution to give optically pure isomers. This resolution can be carried out on any chiral synthetic intermediate or on products of general formula I. Optically pure isomers can also be individually obtained using enantiospecific synthesis. The present invention covers all individual isomers as well as mixtures thereof (for example racemic mixtures or mixtures of diastereomers), whether obtained by synthesis or by physically mixing them.

The compounds of formula I can be obtained by following the processes described below. As it will be obvious to one skilled in the art, the exact method used to prepare a given compound may vary depending on its chemical structure. Moreover, in some of the processes described below it may be necessary or advisable to protect the reactive or labile groups by conventional protective groups. Both the nature of these protective groups and the procedures for their introduction or removal are well known in the art (see for example Greene T. W. and Wuts P. G. M, “Protective Groups in Organic Synthesis”, John Wiley & Sons, 3^{rd} edition, 1999). As an example, as protective groups of an amino function tert-butoxycarbonyl (Boc) or benzyl (Bn) groups can be used. The carboxyl groups can be protected for example in the form of C₁₋₆ alkyl esters or arylalkyl esters, such as benzyl, while the hydroxyl groups can be protected for example with tetrahydropyranyl (THP) groups. Whenever a protective group is present, a later deprotection step will be required, which can be performed under standard conditions in organic synthesis, such as those described in the above-mentioned reference.

Unless otherwise stated, in the methods described below the meanings of the differents substituents are the meanings described above with regard to a compound of general formula I.

The compounds of formula I wherein A represents C (that is, a compound Ia) can be obtained in general by reacting an aldehyde of formula II with a heterocyclic amine of formula III and a compound of formula IV, as shown in the following scheme:

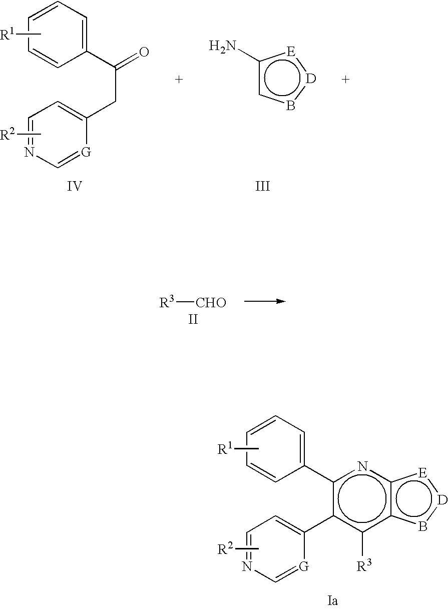

wherein G, B, D, E, R¹, R² and R³ have the meaning described above in connection with a compound of general formula I. This reaction can be carried out preferably in the presence of an acid such as an inorganic acid, for example hydrochloric acid, in a suitable polar solvent such as for example 2-methoxyethanol or ethanol, and heating, preferably at reflux. In certain cases, a dihydropyridine intermediate may be obtained, which can be readily converted into a compound Ia by oxidation with a suitable oxidizing reagent such as cerium (IV) ammonium nitrate.

The compounds II and III are commercially available or can be prepared by methods widely described in the literature.

The compounds of formula IV can be prepared by reacting a compound of formula V with a compound of formula VI

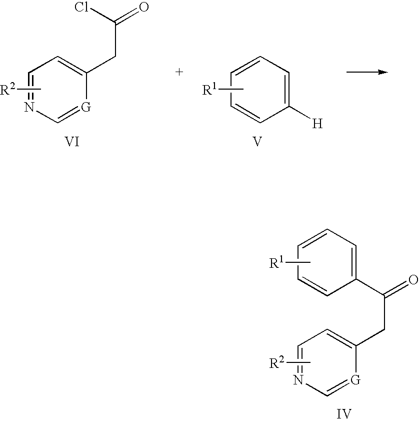

wherein G, R¹ and R² have the meaning described above, in the presence of a Lewis acid, such as AlCl₃, in a suitable halogenated solvent such as dichloromethane.

Alternatively, the compounds of formula IV can be conveniently prepared by reacting a compound of formula VII with a compound of formula VII

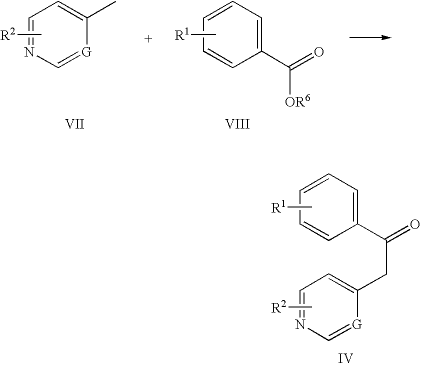

wherein G, R¹ and R² have the meaning described above and R⁶ represents C₁₋₆alkyl, in the presence of a base such as sodium hexamethyldisilazide, in an aprotic polar solvent such as tetrahydrofuran and at a suitable temperature, preferably room temperature.

Alternatively, the compounds of formula IV can be conveniently prepared by reacting a compound of formula VII with a compound of formula IX

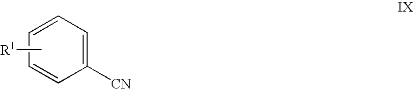

wherein R¹ has the meaning described above, in the presence of a base such as lithium diisopropylamidure, obtained from butyl lythium and N,N′-diisopropylamine, in an aprotic polar solvent such as tetrahydrofuran and cooling, preferably at −78° C.

Alternatively, the compounds of formula IV can be conveniently prepared by reacting a compound of formula VII with a compound of formula X under the same conditions described above to react a compound of formula VII with a compound of formula IX.

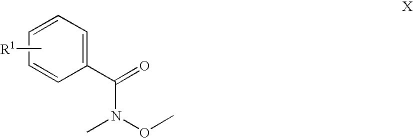

The compounds of formula VI are commercially available or can be readily prepared from the corresponding carboxylic acid by conventional processes.

The compounds V, VII, VII and IX are commercially available or can be prepared by methods widely described in the literature.

The compounds of formula X can be conveniently prepared by reacting a compound of formula XI

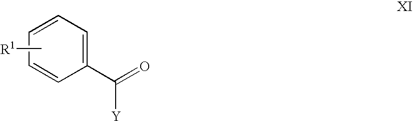

wherein R¹ has the meaning described above and Y represents halogen, preferably Cl, with N,O-dimethylhydroxylamine hydrochloride in the presence of a base such as triethylamine in a suitable halogenated solvent such as for example dichloromethane and cooling preferably at 0° C.

Alternatively, the compounds of formula X can be conveniently prepared by reacting a compound of formula XII

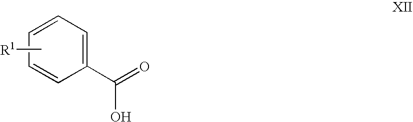

wherein R¹ has the meaning described above, with N,O-dimethylhydroxylamine hydrochloride in the presence of a suitable condensing agent such as for example N-(3-dimethylaminopropyl)-N′-ethylcarbodiimide or dicyclohexylcarbodiimide optionally in the presence of 1-hydroxybenzotriazole, or in the presence of a suitable base, such as pyridine, in a suitable solvent, such as dimethylformamide.

The compounds of formula XI are commercially available or can be prepared by standard reactions starting from the corresponding carboxylic acids of formula XII.

The acids of formula XII are commercially available or can be prepared by methods widely described in the literature, and can be conveniently protected.

Alternatively, the compounds of formula Ia wherein R³═H (i.e. a compound of formula Ia′) can be obtained by reaction of a propenone of formula XIII with a heterocyclic amine of formula III, as shown in the following scheme:

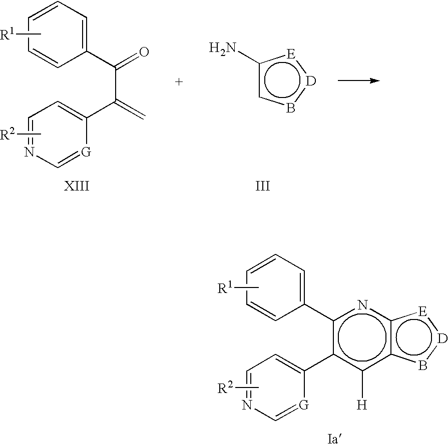

wherein G, B, D, E, R¹ and R² have the meaning described above. The reaction can be carried out in a suitable polar solvent, at an appropriate temperature comprised between room temperature and the boiling point of the solvent and in the presence of an acid. Depending on the pattern of substitution, an extra in situ step of oxidation may be required; this step can be carried out in the same solvent at room temperature by using a suitable oxidizing reagent. Preferably the reaction of XII with III is carried out using ethanol as solvent, at room temperature, in the presence of hydrochloric acid and using cerium (IV) ammonium nitrate as an oxidizing reagent added in situ.

Compounds of formula XIII can be prepared from a compound of formula IV, as shown in the following scheme:

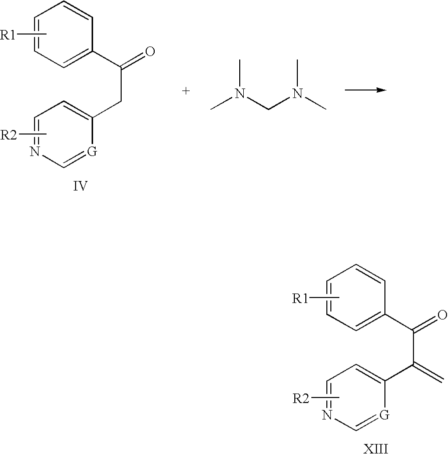

Alternatively, the compounds of formula Ia′ can be obtained in two steps from a compound of formula IV by condensation with a suitable aldehyde XIV to form the intermediate XV, followed by deprotection of the amino group and ring closure, as shown in the following scheme:

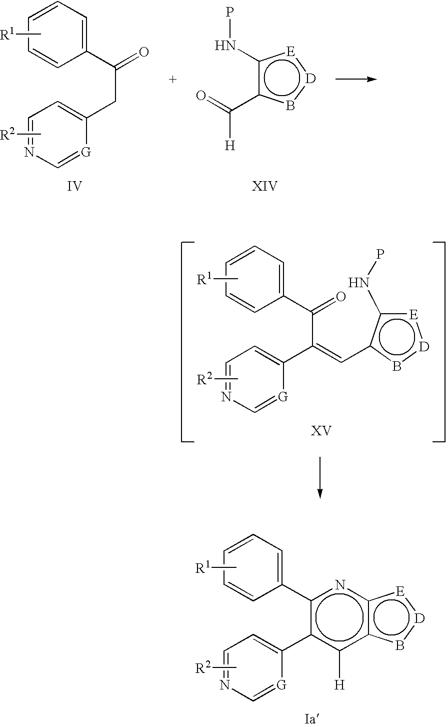

wherein G, B, D, E, R¹ and R² have the meaning described above and P is an amino-protecting group such as the tert-butoxycarbonyl group. This reaction is carried out preferably in the presence of an acid, in a suitable polar solvent such as ethanol, and heating, preferably to reflux.

Compounds of formula XIV can be prepared by different methods described in the literature. For example, they can be obtained from a compound of formula III by protection of the amino group with a suitable amino-protecting group P, for example by treatment with Boc₂O, to form the intermediate XVI and subsequent selective lithiation followed by treatment with dimethylformamide, as shown in the following scheme:

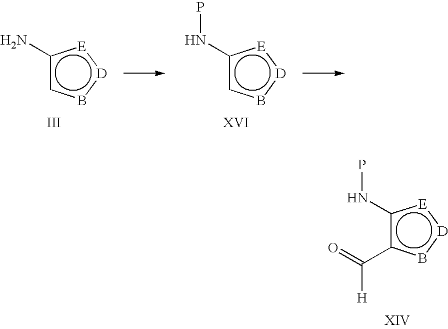

Alternatively, certain compounds of formula Ia′ wherein B═N and D=CR⁴ can be obtained from a compound of formula XVII by condensation under suitable conditions, as shown in the following scheme:

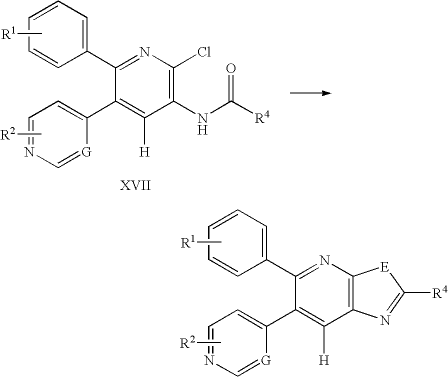

wherein G, E, R¹, R² and R⁴ have the meaning described above.

Compounds of formula XVII can be prepared by acylation of an amine of formula XVIII under standard conditions. The amines of formula XVIII in its turn can be obtained from an acid of formula XIX by Curtius rearrangement under the standard conditions, as shown in the following scheme:

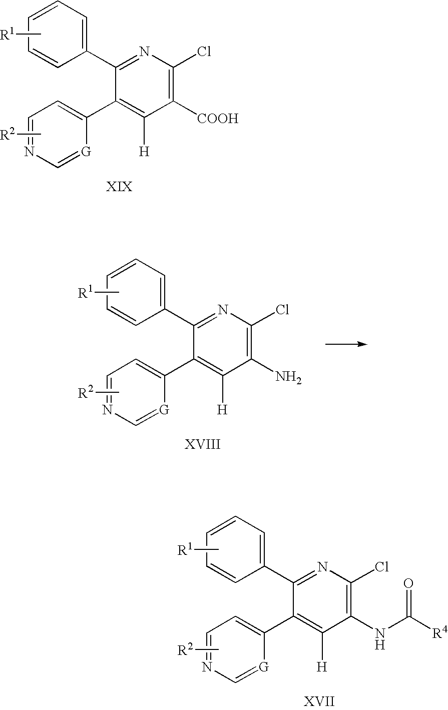

wherein G, R¹, R² and R⁴ have the meaning described above.

Acids of formula XIX can be obtained by simultaneous chlorination and nitrile hydrolysis of intermediate XX with a chlorinating agent such as POCl₃ or PCl₃ without solvent or in a suitable solvent such as dimethylformamide and heating, preferably to reflux, followed by treatment with water.

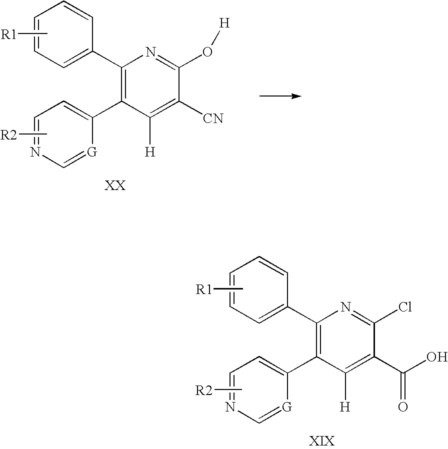

Compounds of formula XX are generally obtained by reaction of a compound of formula XXI with 2-cyanoacetamide, as shown in the following scheme:

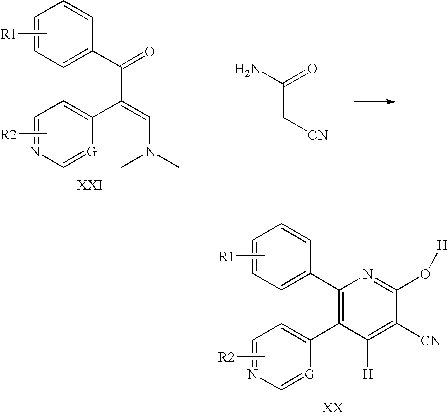

wherein G, R¹ and R² have the meaning described above. This reaction is carried out in the presence of a base such as sodium methoxide, in a suitable solvent such as dimethylformamide and heating, preferably to reflux.

Compounds of formula XXI can be conveniently prepared by reaction of a compound of formula IV with N-(dimethoxymethyl)-N,N-dimethylamine, in a suitable solvent such as tetrahydrofuran.

The compounds of formula I wherein A represents N and R³ represents a group identical to the phenyl substituted with R¹ placed on the adjacent position to the N atom of the 6-membered ring of the central bicyclic moiety (that is, a compound Ib) can in general also be prepared by reacting a compound of formula XXII with a heterocyclic amine of formula XXIII, as shown in the following scheme:

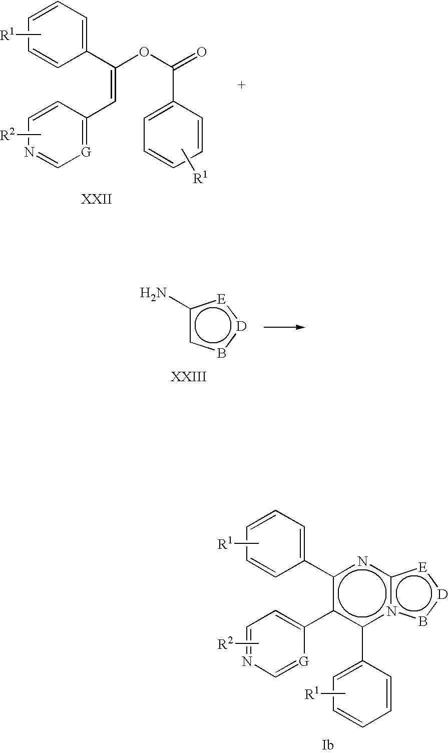

wherein G, R¹, R², B, D and E have the meaning described above. This reaction can be preferably carried out in the presence of an inorganic acid such as for example hydrochloric acid, in a suitable polar solvent such as for example 2-methoxyethanol or ethanol, and heating, preferably at reflux.

The amines of formula XXIII are commercially available or can be prepared by methods widely described in the literature, and can be conveniently protected.

The enol ethers of formula XXII can be prepared by reacting a ketone of formula IV with a compound of formula XI wherein Y represents halogen, preferably Cl, in the presence of a base, such as for example NaH, in a suitable polar solvent such as for example dimethylformamide.

Furthermore, some compounds of the present invention can also be obtained from other compounds of formula I by appropriate conversion reactions of functional groups in one or several steps, using well-known reactions in organic chemistry under the standard experimental conditions.

Thus, for instance, a R⁴ group can be transformed into another R⁴ group, giving rise to new compounds of formula I. For example, R⁴═H can be transformed into R⁴=Br by reaction with a suitable brominating agent, such as Br₂, in a suitable solvent such as chloroform, and at a suitable temperature comprised between room temperature and the boiling point of the solvent;

or R⁴═H can be transformed into R⁴=Cl by reaction with a suitable chlorinating agent, such as N-chlorosuccinimide, in a suitable solvent such as dimethylformamide and at a suitable temperature comprised between room temperature and the boiling point of the solvent;

or R⁴═NH₂ can be transformed into R⁴=halogen by forming a diazonium salt with NaNO₂ followed by reaction with a copper halide, such as CuBr or CuCl, in the presence of an acid, such as for example HBr or HCl;

or R⁴═NH₂ can be transformed into R⁴═H by forming a diazonium salt with NaNO₂ followed by reaction with H₃PO₂, in a suitable solvent such as water;

or R⁴=ester can be transformed into R⁴=dialkylhydroxymethyl or alkanoyl by reaction with a Grignard reagent such as for example methylmagnesium chloride, in a suitable solvent such as tertrahydrofuran;

or R⁴=halogen can be transformed into R⁴═CN by reaction with a cyanide salt such as CuCN in a suitable solvent such as N-methylpyrrolidone and heating, preferably at reflux.

Other conversions upon R⁴, which can also be applied to R², R³ and/or R⁵ to produce other compounds of formula I include, for example:

the conversion of CN into CONH₂ by hydrolysis with a base such as KOH in a suitable solvent such as tert-butanol and heating, preferably at reflux;

the conversion of CN into CH₂NH₂ by reaction with a reducing agent, such as LiAlH₄, in a suitable solvent such as diethyl ether;

the conversion of a carboxylic acid into an ester or an amide by reaction with an alcohol or an amine respectively, in the presence of an activating agent such as N,N′-dicyclohexylcarbodiimide and 1-hydroxybenzotriazole and in a suitable solvent such as dimethylformamide; or alternatively, conversion of a carboxylic acid into an acyl chloride under standard conditions in organic synthesis and subsequent conversion of the latter into an ester or an amide by reaction with an alcohol or an amine respectively, in the presence of a base such as triethylamine, in a suitable solvent such as for example dichloromethane or ethanol, and cooling, preferably at 0° C.;

the conversion of an ester group into a carboxylic acid by hydrolysis in the presence of a base, such as KOH, in a suitable solvent such as ethanol;

the decarboxylation of a carboxylic acid by heating at high temperature and preferably without any solvent;

the conversion of a carboxylic acid group into an amino group by reaction with diphenylphosphorylazide, in the presence of a base, such as for example triethylamine, in a suitable solvent, such as dimethylformamide and at a suitable temperature, preferably room temperature, followed by aqueous treatment at a suitable temperature, preferably 100° C.;

the conversion of OH, SH or NH₂ into OR, SR and NHR or NRR, respectively, by reaction with an alkylating agent R—X, wherein R represents R^{a}, R^{b}, R^{d}, R^{e}, R^{g} or R^{h}; R^{a}, R^{b}, R^{d}, R^{e}, R^{g} and R^{h} have the meaning described in general formula I and X represents halogen, preferably chloro or bromo, in the presence of a base such as triethylamine, sodium hydroxide, sodium carbonate, potassium carbonate or sodium hydride, among others, in a suitable solvent such as dichloromethane, chloroform, dimethylformamide or toluene, and at a temperature comprised between room temperature and the boiling point of the solvent;

alternatively, NHR can be transformed into NCH₃R, wherein R represents R^{a}, R^{b}, R^{d}, R^{e}, R^{g} or R^{h} and R^{a}, R^{b}, R^{d}, R^{e}, R^{g} and R^{h} have the meaning described in general formula I, by reaction with formaldehyde in acid medium, such as formic acid and preferably heating;

the conversion of an amine into an amide group by reaction with a carboxylic acid in the presence of a suitable condensing agent such as for example N-(3-dimethylaminopropyl)-N′-ethylcarbodiimide or dicyclohexylcarbodiimide optionally in the presence of 1-hydroxybenzotriazole, or in the presence of a suitable base such as pyridine, in a suitable solvent, such as dimethylformamide; or alternatively an amine can be transformed into an amide group by reaction with an acyl chloride in the presence of a base such as triethylamine in a suitable solvent such as for example dichloromethane, and cooling preferably at 0° C.;

the conversion of an amine into a urea or a carbamate by a two step sequence that involves reacting the amine with an activating agent such as triphosgene, in the presence of a base such as diisopropylethylamine, triethylamine or N-methylmorpholine, in a suitable solvent such as acetonitrile or a halogenated hydrocarbon such as chloroform or dichloromethane, and then reacting the resulting compound with the second amine in the case of the urea or with an alcohol in the case of the carbamate, in a suitable solvent, such as the solvent used in the first step; or alternatively an amine can be transformed into a urea or carbamate by reaction with an isocyanate or a chloroformate, respectively, in a suitable solvent, such as for example dimethylformamide, and at a suitable temperature, preferably room temperature;

the conversion of an amine into a sulfonamide group by reaction with a sulfonyl halide, such as sulfonyl chloride, optionally in the presence of a base such as dimethylaminopyridine, in a suitable solvent such as for example dioxane, chloroform, dichloromethane or pyridine;

the conversion of a hydroxyl group into an ester group by reaction with a carboxylic acid under the standard conditions previously mentioned;

the conversion of a sulfanyl group into a sulfinyl or sulfonyl group by reaction with 1 or 2 equivalents, respectively, of a suitable oxidizing agent such as m-chloroperbenzoic acid in a suitable solvent such as for example dichloromethane;

alternatively, the conversion of a sulfanyl group into a sulfinyl or sulfonyl group can be carried out in the presence of NaWO₄ and H₂O₂ in a water-acetic acid mixture and preferably heating;

the conversion of a primary or secondary hydroxyl group into a leaving group, for example an alkylsulfonate or arylsulfonate such as mesylate or tosylate or a halogen such as Cl, Br or I, by reaction with a sulfonyl halide, such as methanesulfonyl chloride, in the presence of a base, such as pyridine or triethylamine, in a suitable solvent such as for example dichloromethane or chloroform, or with a halogenating agent, such as for example SOCl₂, in a suitable solvent such as tetrahydrofuran; said leaving group can then be substituted by reaction with an alcohol, amine or thiol, optionally in the presence of a base, such as K₂CO₃ and in a suitable solvent such as dimethylformamide, 1,2-dimethoxyethane or acetonitrile;

the conversion of a primary amide into a secondary amide by reaction with an alkylating agent in the presence of a strong base such as sodium hydride in a suitable solvent and at a temperature comprised between room temperature and the boiling point of the solvent; the conversion of a CHO group into an amine group by reaction with an amine in the presence of a reducing agent such as sodium triacetoxyborohydride, in a suitable solvent such as for example 1,2-dichloroethane or dichloromethane;

the conversion of an acetal group into an aldehyde group by reaction in acidic medium, for example in HCl, at a suitable temperature, preferably at reflux;

the conversion of an ester group into an alcohol group by reaction with a reducing agent, such as LiAlH₄, in a suitable solvent, such as tetrahydrofuran;

the conversion of a sulfonyl group bonded to an aromatic ring by displacement with an amine to give the corresponding amino derivative or with an alcohol to give the corresponding alkoxy derivative, either in a suitable solvent or without any solvent and heating, preferably at a temperature comprised between room temperature and 100° C.;

the conversion of halogen into a NHR group, wherein R represents R^{a}, R^{b}, R^{d}, R^{e}, R^{g} or R^{h} and wherein R^{a}, R^{b}, R^{d}, R^{e}, R^{g} and R^{h} have the meaning described in general formula I, by reaction with an amine of formula H₂NR and preferably heating;

alternatively, a halogen group can be transformed into a NHR group by reaction with an amine of formula H₂NR wherein R represents R^{a}, R^{b}, R^{d}, R^{e}, R^{g} or R^{h} and wherein R^{a}, R^{b}, R^{d}, R^{e}, R^{g} and R^{h} have the meaning described in general formula I, in the presence of a base, such as Cs₂CO₃ or sodium tert-butoxide, in the presence of a palladium catalyst, such as palladium acetate (II), and a phosphine such as 2,2′-bis(diphenylphosphino)-1,1′-binaphthyl, in a solvent, such as toluene, and preferably heating; and

the conversion of a halogen group into a phenyl or heteroaryl group by treatment with a phenyl- or a heteroarylboronic acid in the presence of a catalyst, such as for example a palladium catalyst such as palladium acetate (II) or Pd(PPh₃)₄, and of a base such as Na₂CO₃, K₂CO₃ or CsF, in a suitable polar solvent, such as 1,2-dimethoxyethane or toluene-water mixtures, and preferably heating.

Likewise, any of the aromatic rings of the compounds of the present invention can undergo electrophilic aromatic substitution reactions, widely described in the literature.

Some of these interconversion reactions are explained in greater detail in the examples.

As it will be obvious to those skilled in the art, these interconversion reactions can be carried out upon the compounds of formula I as well as upon any suitable synthesis intermediate thereof.

As mentioned previously, the compounds of the present invention act as p38 kinase inhibitors, inducing the reduction of proinflammatory cytokines. Therefore, the compounds of the invention are expected to be useful to treat or prevent diseases in which p38 plays a role in mammals, including human beings. This includes diseases caused by overproduction of cytokines such as TNF-α, IL-1, IL-6 or IL-8. These diseases include, but are not limited to, immune, autoimmune and inflammatory diseases, cardiovascular diseases, infectious diseases, bone resorption disorders, neurodegenerative diseases, proliferative diseases and processes associated with cyclooxygenase-2 induction.

As an example, immune, autoimmune and inflammatory diseases that can be treated or prevented with the compounds of the present invention include rheumatic diseases (e.g. rheumatoid arthritis, psoriatic arthritis, infectious arthritis, progressive chronic arthritis, deforming arthritis, osteoarthritis, traumatic arthritis, gouty arthritis, Reiter's syndrome, polychondritis, acute synovitis and spondylitis), glomerulonephritis (with or without nephrotic syndrome), autoimmune hematologic disorders (e.g. hemolytic anemia, aplasic anemia, idiopathic thrombocytopenia and neutropenia), autoimmune gastritis and autoimmune inflammatory bowel diseases (e.g. ulcerative colitis and Crohn's disease), host versus graft disease, allograft rejection, chronic thyroiditis, Graves' disease, schleroderma, diabetes (type I and type II), active hepatitis (acute and chronic), primary biliary cirrhosis, myasthenia gravis, multiple sclerosis, systemic lupus erythematosus, psoriasis, atopic dermatitis, contact dermatitis, eczema, skin sunburns, chronic renal insufficiency, Stevens-Johnson syndrome, idiopathic sprue, sarcoidosis, Guillain-Barré syndrome, uveitis, conjunctivitis, keratoconjunctivitis, otitis media, periodontal disease, pulmonary interstitial fibrosis, asthma, bronchitis, rhinitis, sinusitis, pneumoconiosis, pulmonary insufficiency syndrome, pulmonary emphysema, pulmonary fibrosis, silicosis, chronic inflammatory pulmonary disease (e.g. chronic obstructive pulmonary disease) and other inflammatory or obstructive diseases of the airways.

Cardiovascular diseases that can be treated or prevented include, among others, myocardial infarction, cardiac hypertrophy, cardiac insufficiency, ischaemia-reperfusion disorders, thrombosis, thrombin-induced platelet aggregation, acute coronary syndromes, atherosclerosis and cerebrovascular accidents.

Infectious diseases that can be treated or prevented include, among others, sepsis, septic shock, endotoxic shock, sepsis by Gram-negative bacteria, shigellosis, meningitis, cerebral malaria, pneumonia, tuberculosis, viral myocarditis, viral hepatitis (hepatitis A, hepatitis B and hepatitis C), HIV infection, retinitis caused by cytomegalovirus, influenza, herpes, treatment of infections associated with severe burns, myalgias caused by infections, cachexia secondary to infections, and veterinary viral infections such as lentivirus, caprine arthritic virus, visna-maedi virus, feline immunodeficiency virus, bovine immunodeficiency virus or canine immunodeficiency virus.

Bone resorption disorders that can be treated or prevented include osteoporosis, osteoarthritis, traumatic arthritis and gouty arthritis, as well as bone disorders related with multiple myeloma, bone fracture and bone grafting and, in general, all these processes wherein it is necessary to induce osteoblastic activity and increase bone mass.

Neurodegenerative diseases that can be treated or prevented include Alzheimer's disease, Parkinson's disease, cerebral ischaemia and traumatic neurodegenerative disease, among others.

Proliferative diseases that can be treated or prevented include endometriosis, solid tumors, acute and chronic myeloid leukemia, Kaposi sarcoma, multiple myeloma, metastatic melanoma and angiogenic disorders such as ocular neovascularisation and infantile haemangioma.

p38 kinase inhibitors also inhibit the expression of proinflammatory proteins such as cyclooxygenase-2 (COX-2), the enzyme responsible for prostaglandin production. Therefore, the compounds of the present invention can also be used to treat or prevent diseases mediated by COX-2 and especially to treat processes with edema, fever and neuromuscular pain such as cephalea, pain caused by cancer, tooth pain, arthritic pain, hyperalgesia and allodynia.

In vitro and in vivo assays to determine the ability of a compound to inhibit p38 activity are well known in the art. For example, a compound to be tested can be contacted with the purified p38 enzyme to determine whether inhibition of p38 activity occurs. Alternatively, cell-based assays can be used to measure the ability of a compound to inhibit the production of cytokines such as TNFalpha, e.g. in stimulated peripheral blood mononuclear cells (PBMCs) or other cell types. Detailed disclosure of an assay that can be used to test the biological activity of the compounds of the invention as p38 inhibitors can be found below (see Example 57).

For selecting active compounds, testing at 10 μM must result in an activity of more than 50% inhibition in the test provided in Example 57. More preferably, compounds should exhibit more than 50% inhibition at 1 μM, and still more preferably, they should exhibit more than 50% inhibition at 0.1 μM.

The present invention also relates to a pharmaceutical composition which comprises a compound of the present invention (or a pharmaceutically acceptable salt or solvate thereof) and one or more pharmaceutically acceptable excipients. The excipients must be “acceptable” in the sense of being compatible with the other ingredients of the composition and not deleterious to the recipients thereof.

The compounds of the present invention can be administered in the form of any pharmaceutical formulation, the nature of which, as it is well known, will depend upon the nature of the active compound and its route of administration. Any route of administration may be used, for example oral, parenteral, nasal, ocular, rectal and topical administration.

Solid compositions for oral administration include tablets, granulates and capsules. In any case the manufacturing method is based on a simple mixture, dry granulation or wet granulation of the active compound with excipients. These excipients can be, for example, diluents such as lactose, microcrystalline cellulose, mannitol or calcium hydrogenphosphate; binding agents such as for example starch, gelatin or povidone; disintegrants such as sodium carboxymethyl starch or sodium croscarmellose; and lubricating agents such as for example magnesium stearate, stearic acid or talc. Tablets can be additionally coated with suitable excipients by using known techniques with the purpose of delaying their disintegration and absorption in the gastrointestinal tract and thereby provide a sustained action over a longer period, or simply to improve their organoleptic properties or their stability. The active compound can also be incorporated by coating onto inert pellets using natural or synthetic film-coating agents. Soft gelatin capsules are also possible, in which the active compound is mixed with water or an oily medium, for example coconut oil, mineral oil or olive oil.

Powders and granulates for the preparation of oral suspensions by the addition of water can be obtained by mixing the active compound with dispersing or wetting agents; suspending agents and preservatives. Other excipients can also be added, for example sweetening, flavouring and colouring agents.

Liquid forms for oral administration include emulsions, solutions, suspensions, syrups and elixirs containing commonly-used inert diluents, such as purified water, ethanol, sorbitol, glycerol, polyethylene glycols (macrogols) and propylene glycol. Said compositions can also contain coadjuvants such as wetting, suspending, sweetening, flavouring agents, preservatives and buffers.

Injectable preparations, according to the present invention, for parenteral administration, comprise sterile solutions, suspensions or emulsions, in an aqueous or non-aqueous solvent such as propylene glycol, polyethylene glycol or vegetable oils. These compositions can also contain coadjuvants, such as wetting, emulsifying, dispersing agents and preservatives. They may be sterilized by any known method or prepared as sterile solid compositions which will be dissolved in water or any other sterile injectable medium immediately before use. It is also possible to start from sterile materials and keep them under these conditions throughout all the manufacturing process.

For the rectal administration, the active compound can be preferably formulated as a suppository on an oily base, such as for example vegetable oils or solid semisynthetic glycerides, or on a hydrophilic base such as polyethylene glycols (macrogol).

The compounds of the invention can also be formulated for their topical application for the treatment of pathologies occurring in zones or organs accessible through this route, such as eyes, skin and the intestinal tract. Formulations include creams, lotions, gels, powders, solutions and patches wherein the compound is dispersed or dissolved in suitable excipients.

For the nasal administration or for inhalation, the compound can be formulated as an aerosol and it can be conveniently released using suitable propellants.

The dosage and frequency of doses will depend upon the nature and severity of the disease to be treated, the age, the general condition and body weight of the patient, as well as the particular compound administered and the route of administration, among other factors. A representative example of a suitable dosage range is from about 0.01 mg/Kg to about 100 mg/Kg per day, which can be administered as a single or divided doses.

The invention is illustrated by the following examples.

### EXAMPLES

The following abbreviations have been used:

ACN: acetonitrile
BuLi: n-butyllithium
DMF: dimethylformamide
DMSO: dimethylsulfoxide
EtOAc: ethyl acetate
EtOH: ethanol
KOtBu: potassium tert-butoxide
LC-MS: liquid chromatography-mass spectrometry
MeOH: methanol
NaOMe: sodium methoxide
NH₄OAc: ammonium acetate

### NMM: N-methylmorpholine

### NMP: N-methylpyrrolidone

TEA: triethylamine
TFA: trifluoroacetic acid
THF: tetrahydrofuran
t_{R}: retention time
The following chromatographic methods have been used to perform the LC-MS spectra:
Method 1: Column Tracer Excel 120, ODSB 5 μm (10 mm×0.21 mm), column temperature: 30° C., flow: 0.35 mL/min, eluent: A=ACN, B=0.1% HCOOH, gradient: 0 min 10% A—10 min 90% A.
Method 2: Column X-Terra MS C18 5 μm (150 mm×2.1 mm), column temperature: 30° C., flow: 0.35 mL/min, eluent: A=ACN, B=10 mM NH₄OAc (pH=6.80), gradient: 0 min 10% A—10 min 90% A.
Method 3: Column 3.5 μm X-Terra MS C18 20×4.6 mm; flow: 1 mL/min; detection: 210 nm; column temperature: 40° C.; solvent A: 0.05% TFA in ACN/H₂O=9/1 (v/v); solvent B: 0.05% TFA in H₂O; gradient: solvent A/B=0/100 to 100/0 (v/v) in 5 min.
The following analytical HPLC methods were used for determination of retention time:
Method 4: Column 5 μm Luna C-18(2) 150×4.6 mm; flow: 1 mL/min; detection: 210 nm; column temperature: 40° C.; solvent A: ACN/H₂O=1/9 (v/v); solvent B: ACN; solvent C, 0.1 M aqueous TFA; gradient: solvent A/B/C=77/20/3 to 15/82/3 (v/v/v) in 30 min, then constant for an additional 10 min at A/B/C=15/82/3 (v/v/v).
Method 5: Column 5 μm Luna C-18(2) 150×4.6 mm; flow: 1 mL/min; detection: 210 nm; column temperature: 40° C.; solvent A: 0.1% TFA in ACN/H₂O=1/9 (v/v); solvent B: 0.1% TFA in ACN; gradient: solvent A/B=100/0 to 0/100 (v/v) in 30 min.
Method 6: Column 5 μm Atlantis dC 18 150×4.6 mm; flow: 1 mL/min; detection: 210 nm; column temperature: 40° C.; solvent A: 0.1% TFA in ACN/H₂O=1/9 (v/v); solvent B: 0.1% TFA in ACN; gradient: solvent A/B=100/0 to 0/100 (v/v) in 30 min.
Preparative HPLC have been performed using the following chromatographic conditions:
Luna column 10 m C18(2) [250×50 mm]; eluent: 0.1% TFA solution in ACN/water mixtures of decreasing polarity.
Reactions carried out under microwave irradiation were performed in a Biotage Initiator Microwave Synthesizer. The reaction mixture was set in a sealed tube and heated at a constant temperature (as indicated in each example) under microwave irradiation between 0 and 75 W. After that, the reaction was cooled to room temperature.

### Reference Example 1

### 1-(4-Fluorophenyl)-2-(4-pyridyl)ethanone

### a) Ethyl 4-fluorobenzoate

To a TEA solution (28.4 mL, 211 mmol) in EtOH (143 mL) cooled to 0° C. and under argon atmosphere, 4-fluorobenzoyl chloride (33.50 g, 25 mL) was slowly added and the resulting mixture was stirred at room temperature for 7 h. It was concentrated and EtOAc and water were added to the residue. The phases were separated and the aqueous phase was reextracted with EtOAc. The combined organic extracts were washed with 10% NaHCO₃ aqueous solution, dried over Na₂SO₄ and concentrated to dryness, affording 35.00 g of the desired compound (98% yield).

¹H NMR (300 MHz, CDCl₃) 6 (TMS): 1.39 (t, J=7.2 Hz, 3H), 4.36 (c, J=7.2 Hz, 2H), 7.12 (m, 2H), 8.05 (m, 2H).

### b) Title Compound

To a mixture of 4-methylpyridine (33.60 g, 356.0 mmol) and ethyl 4-fluorobenzoate (60.53 g, 356.0 mmol, obtained in section a) in THF (350 mL) cooled to 10° C., 2 N sodium hexamethyldisilazide (281 mL) was added under argon so that the temperature did not exceed 10° C. Once the addition was finished, the resulting mixture was stirred at room temperature for 18 h. It was cooled to 5-10° C. and water (200 mL) was added. The aqueous phase was separated and extracted twice with EtOAc (200 and 100 mL respectively). The combined organic extracts were washed with water and concentrated. The crude product obtained was purified by recrystallization from EtOAc (40 mL) and cyclohexane (200 mL), affording 38.79 g of the title compound. Mother liquor was purified by column chromatography, affording 10.24 g of the title compound (global yield: 64%).

¹H NMR (300 MHz, CDCl₃) 6 (TMS): 4.29 (s, 2H), 7.14-7.23 (complex signal, 4H), 8.05 (m, 2H), 8.59 (dd, Jₒ=1.6 Hz, Jₘ=4.4 Hz, 2H).

### Reference Example 2

### 1-Phenyl-2-(4-pyridyl)ethanone

A solution of diisopropylamine (22 mL, 15.03 mmol) in THF (200 mL) under argon was cooled to −78° C. Then, BuLi (96 mL of a 1.6 M solution in hexane, 153.0 mmol) was added dropwise. One h later a solution of 4-methylpyridine (15.00 g, 161.1 mmol) in THF (75 mL) was added and the resulting mixture was allowed to warm up to 0° C. It was stirred at this temperature for 30 min. It was then cooled to −78° C., benzonitrile (18.27 g, 177.2 mmol) in THF (75 mL) was added and the resulting mixture was stirred at −78° C. for 2 h. The mixture was stirred at room temperature overnight. Water (225 mL) was added, the mixture was cooled with an ice-water bath and was adjusted to pH 1 with 48% HBr. The organic phase was separated. The aqueous phase was heated at reflux for 2 h, was allowed to cool and was extracted with diethyl ether. The aqueous phase was brought to neutral pH with 1N NaOH and extracted with EtOAc. The organic phase was dried over Na₂SO₄ and concentrated to dryness, affording 28.53 g of the title compound (90% yield).

¹H NMR (300 MHz, CDCl₃) 6 (TMS): 4.29 (s, 2H), 7.20 (dd, Jₒ=1.6 Hz, Jₘ=4.4 Hz, 2H), 7.49 (m, 2H), 7.58 (m, 1H), 8.00 (d, J=8.2 Hz, 2H), 8.56 (dd, Jₒ=1.6 Hz, Jₘ=4.4 Hz, 2H).

### Reference Example 3

### 1-(4-Fluorophenyl)-2-(4-pyridyl)vinyl 4-fluorobenzoate

To a suspension of NaH (0.81 g, 18.6 mmol) in DMF (30 mL) under argon and cooled to 0° C., a solution of 1-(4-fluorophenyl)-2-(4-pyridyl)ethanone (2.00 g, 9.3 mmol, obtained in reference example 1) in DMF (15 mL) was added and the resulting mixture was stirred at room temperature for 30 min. Then, it was cooled to 0° C. and a solution of 4-fluorobenzoyl chloride (2.95 g, 1.9 mmol) in DMF (10 mL) was added. It was stirred at room temperature overnight. Water was added and the solvent was evaporated. The residue was dissolved in a CHCl₃-water mixture and the phases were separated. The aqueous phase was extracted with CHCl₃ (×3). The organic phase was washed twice with water, dried over Na₂SO₄ and concentrated to dryness. The crude product obtained was purified by chromatography on silica gel using hexane-EtOAc mixtures of increasing polarity as eluent, affording 0.98 g of the desired compound as a yellow solid (31% yield).

¹H NMR (300 MHz, CDCl₃) δ (TMS): 6.68 (s, 1H), 7.11 (t, J=8.6 Hz, 2H), 7.29 (t, J=8.6 Hz, 2H), 7.39 (d, J=6.0 Hz, 2H), 7.60 (dd, Jₒ=5.2 Hz, Jₘ=8.8 Hz, 2H), 8.27 (dd, Jₒ=5.4 Hz, Jₘ=8.8 Hz, 2H), 8.58 (d, J=6.0 Hz, 2H).

### Reference Example 4

### 1-Phenyl-2-(4-pyridyl)vinyl benzoate

Following a similar procedure to that described in reference example 3, but using 1-phenyl-2-(4-pyridyl)ethanone (obtained in reference example 2) instead of 1-(4-fluorophenyl)-2-(4-pyridyl)ethanone and benzoyl chloride instead of 4-fluorobenzoyl chloride, the title compound was obtained (62% yield). LC-MS (method 1): t_{R}=7.05 min; m/z=302.1 [M+H]⁺.

### Reference Example 5

### 1-(4-Fluoro-phenyl)-2-(2-methylsulfanyl-pyrimidin-4-yl)-propenone

### a) 4-Methyl-2-(methylsulfanyl)pyrimidine

To a solution of NaOH (7.46 g, 186.4 mmol) in water (120 mL) was added 4-methylpyrimidine-2-thiol hydrochloride (13.78 g, 84.7 mmol) and subsequently iodomethane (13.23 g, 93.2 mmol) was added dropwise under argon atmosphere. It was stirred at room temperature for 2 h and then extracted with CH₂Cl₂ (2×). The organic phase was dried over Na₂SO₄ and concentrated to dryness. The crude product obtained was purified by chromatography on silica gel using hexane-EtOAc mixtures of increasing polarity as eluent, to afford 10.26 g of the desired compound (yield: 86%).

### b) 1-(4-Fluoro-phenyl)-2-(2-methylsulfanyl-pyrimidin-4-yl)-ethanone

To a solution of 4-methyl-2-(methylsulfanyl)pyrimidine (21.00 g, 150.0 mmol) and ethyl 4-fluorobenzoate (25.14 g, 150.0 mmol) in THF (300 mL) under argon atmosphere, a solution of sodium hexamethyldisilazide (150 mL of a 2 M solution in THF, 300 mmol) in THF (150 mL) was added dropwise while cooling with an ice-bath. It was stirred at room temperature for 2 h. Saturated NH₄Cl solution was added and the solvent was evaporated. The residue was taken up in a mixture of EtOAc and water and the phases were separated. The aqueous phase was extracted with EtOAc. The combined organic phases were washed with brine, dried over Na₂SO₄ and concentrated to dryness, to afford 36.36 g of the title compound (yield: 93%).

### c) 1-(4-Fluoro-phenyl)-2-(2-methylsulfanyl-pyrimidin-4-yl)-propenone

To a solution of N,N,N′,N′-tetramethyl-methanediamine (0.421 mL, 3.05 mmol) in dry CH₂Cl₂ (2.5 mL), a solution of 1-(4-fluoro-phenyl)-2-(2-methylsulfanyl-pyrimidin-4-yl)-ethanone (0.5 g, 1.91 mmol) and acetic anhydride (0.397 mL, 4.20 mmol) in dry CH₂Cl₂ (5 mL) was added dropwise at −15° C. The reaction was stirred at that temperature under nitrogen atmosphere for 10 min. After that a mixture of diethylether/water (1:1) was added. The organic phase was washed with water (2×) and brine (2×), dried over MgSO₄ and concentrated to dryness to afford 483 mg of the title product as a colorless oil (yield: 92%).

MS: m/z=275 [M+H]⁺.

### Reference Example 6

### 1-(4-Methoxy-phenyl)-2-(2-methylsulfanyl-pyrimidin-4-yl)-propenone

### a) 1-(4-Methoxy-phenyl)-2-(2-methylsulfanyl-pyrimidin-4-yl)-ethanone

Following a similar procedure to that described in reference example 5b, but using ethyl 4-methoxybenzoate instead of ethyl 4-fluorobenzoate, the title compound was obtained (7.2 g, yield: 87%).

HPLC (method 6): t_{R}=20.45 min; MS: m/z=275 [M+H]⁺.

### b) 1-(4-Methoxy-phenyl)-2-(2-methylsulfanyl-pyrimidin-4-yl)-propenone

Following a similar procedure to that described in reference example 5c, but using 1-(4-methoxy-phenyl)-2-(2-methylsulfanyl-pyrimidin-4-yl)-ethanone instead of 1-(4-fluoro-phenyl)-2-(2-methylsulfanyl-pyrimidin-4-yl)-ethanone, the title compound was obtained (320 mg, yield: 80%).

HPLC (method 6): t_{R}=21.14 min; MS: m/z 287 [M+H]⁺.

### Reference Example 7

### 4-Amino-1H-pyrazole-3-carboxylic acid methyl ester

To a solution of 4-nitro-1H-pyrazole-3-carboxylic acid methyl ester (1.3 g, 7.6 mmol) in MeOH (100 mL) were added ammonium formate (3.35 g, 53.2 mmol) and 5% palladium on carbon (225 mg). The reaction was stirred for 17 h at room temperature under a nitrogen atmosphere. Removal of the catalyst by filtration, followed by evaporation of the solvent afforded the crude title compound as a brown solid (yield: 95%).

### Reference Example 8

### (4-Formyl-5-methyl-isoxazol-3-yl)-carbamic acid tert-butyl ester

### a) (5-Methyl-isoxazol-3-yl)-carbamic acid tert-butyl ester

To a solution of 3-amino-5-methylisoxazole (5 g, 51 mmol) in pyridine (80 mL), di-tert-butyl dicarbonate (11.1 g, 51 mmol) was added at room temperature. The reaction was stirred overnight. NaOH aq. in MeOH was added and stirred for 3 h at room temperature. EtOAc and water were added and the phases were separated. The aqueous phase was extracted with EtOAc. The combined organic phases were dried over Na₂SO₄ and concentrated to dryness. The crude product obtained was purified by chromatography on silica gel using heptane-EtOAc mixtures of increasing polarity as eluent, to afford 6.44 g of the title compound (yield: 63%)

### b) (4-Formyl-5-methyl-isoxazol-3-yl)-carbamic acid tert-butyl ester

To a solution of (5-methyl-isoxazol-3-yl)-carbamic acid tert-butyl ester (2 g, 10.1 mmol, obtained in reference example 8a) in THF (50 mL), BuLi (1.6 M solution in hexane, 14.5 mL, 23.2 mmol) was added at −78° C. and under N₂ atmosphere. The reaction mixture was stirred for 30 min at −78° C. and then for 30 min at room temperature. After cooling down to −78° C., DMF (2 mL, 24.2 mmol) was added and the reaction mixture was stirred for 2 h at room temperature. EtOAc and water were added and the phases were separated. The aqueous phase was extracted with EtOAc. The combined organic phases were washed with water, dried over Na₂SO₄ and concentrated to dryness. The crude product was purified by chromatography on silica gel using heptane-EtOAc mixtures of increasing polarity as eluent, to afford 498 mg of the desired compound (yield: 22%).

### Reference Example 9

### 2-Pyridin-4-yl-1-(3-trifluoromethyl-phenyl)-ethanone

### a) N-Methoxy-N-methyl-3-(trifluoromethyl)benzamide

In a volumetric flask N,O-dimethylhydroxylamine hydrochloride (7.62 g, 70 mmol) and CH₂Cl₂ (135 mL) were introduced under nitrogen atmosphere at 0° C. 3-(trifluoromethyl)benzoyl chloride (14.81 g, 71 mmol) was added followed by the slow addition of TEA (15.81 g, 156.2 mmol). The reaction was stirred for 30 min at 5° C. and allowed to reach room temperature. It was washed with 5% aqueous citric acid (60 mL) and with 5% aqueous NaHCO₃ (60 mL). The aqueous phase was extracted with CH₂Cl₂. The organic phase was dried over Na₂SO₄ and concentrated to dryness, to afford 16.8 g of the desired compound (yield: 100%).

### b) 2-Pyridin-4-yl-1-(3-trifluoromethyl-phenyl)-ethanone

To a solution of diisopropylamine (15.3 mL, 108 mmol) in THF (170 mL), cooled to −78° C., BuLi (68 mL of a 1.6 M solution in hexane, 108 mmol) was added dropwise and under nitrogen atmosphere. After 5 min, the reaction was allowed to reach −30° C. and then stirred at this temperature for 30 min. At this temperature a solution of 4-methylpyridine (7.07 mL, 72.1 mmol) in THF (57 mL) was added over 20 min. The mixture was stirred at 0° C. for 15 min and a solution of N-methoxy-N-methyl-3-(trifluoromethyl)benzamide (obtained in section a) in THF (57 mL) was added over 30 min. The reaction was allowed to reach room temperature. Water (100 mL) and EtOAc (100 mL) were added and the mixture was stirred for 30 min. The organic phase was separated, dried over Na₂SO₄ and concentrated to dryness, to afford 16.2 g of the desired compound (yield: 76%).

### Reference Example 10

### N-[2-Chloro-6-(4-fluoro-phenyl)-5-(2-methylsulfanyl-pyrimidin-4-yl)-pyridin-3-yl]-acetamide

### a) 3-(Dimethylamino)-1-(4-fluorophenyl)-2-[2-(methylsulfanyl)pyrimidin-4-yl]prop-2-en-1-one

To a solution of 1-(4-fluoro-phenyl)-2-(2-methylsulfanyl-pyrimidin-4-yl)-ethanone (37.8 g, 144 mmol, obtained in reference example 5b) in anhydrous THF (500 mL), dimethylformamide dimethyl acetal (27.7 g, 328 mmol) was added under nitrogen atmosphere. The reaction mixture was stirred overnight at room temperature. The solvent was evaporated to afford 49.14 g of the title compound (yield: quantitative).

### b) 6-(4-Fluorophenyl)-2-(hydroxy)-5-(2-methylsulfanylpyrimidin-4-yl)pyridine-3-carbonitrile

To a solution of 3-(dimethylamino)-1-(4-fluorophenyl)-2-[2-(methylsulfanyl)pyrimidin-4-yl]prop-2-en-1-one (4.68 g, 14.7 mmol, obtained in reference example 10a) in DMF (60 mL), 2-cyanoacetamide (1.42 g, 16.9 mmol) was added under nitrogen atmosphere. Then, NaOMe (1.75 g, 32.4 mmol) was added and the mixture was heated to reflux for 1 h. It was allowed to cool, concentrated and diluted with water. The pH was adjusted to 4 with 1 N HCl. The crude product was purified by chromatography on silica gel using heptane-EtOAc mixtures of increasing polarity as eluent, to afford 2.95 g of the desired compound (yield: 59%).

### c) 2-Chloro-6-(4-fluorophenyl)-5-(2-methylsulfanyl-pyrimidin-4-yl)-nicotinic acid

To a solution of 6-(4-fluorophenyl)-2-(hydroxy)-5-(2-methylsulfanylpyrimidin-4-yl)pyridine-3-carbonitrile (1.10 g, 3.25 mmol, obtained in reference example 10b) in DMF (2.5 mL), phosphorus oxychloride (4 mL) was added at room temperature and under nitrogen atmosphere. The mixture was heated to reflux and stirred for 4 h. The mixture was then cooled to room temperature, poured into ice water and extracted with EtOAc (2×). The combined organic phases were washed with 0.2 M NaOH solution and the layers were separated. The aqueous phase was acidified with 2 M HCl solution and subsequently extracted with EtOAc (2×). The combined organic phases were washed with brine (1×), dried over Na₂SO₄ and concentrated to dryness, to afford 0.91 g of the title compound (yield: 75%)

MS: m/z=376 [M+H]⁺.

### d) 2-Chloro-6-(4-fluoro-phenyl)-5-(2-methylsulfanyl-pyrimidin-4-yl)-pyridin-3-ylamine

To a solution of 2-chloro-6-(4-fluorophenyl)-5-(2-methylsulfanyl-pyrimidin-4-yl)-nicotinic acid (0.91 g, 2.42 mmol, obtained in reference example 10c) in NMP (12 mL), TEA (0.43 mL, 3.15 mmol) and diphenylphosphorylazide (0.57 mL, 2.66 mmol) were subsequently added at room temperature and under nitrogen atmosphere. The mixture was heated to 90° C. and stirred for 2 h. It was then cooled to room temperature and NaHCO₃ solution was added, which was extracted with EtOAc (2×). The combined organic phases were washed with NaHCO₃ solution (1×) and brine (1×), dried over Na₂SO₄ and concentrated to dryness to afford 0.75 g of the title compound (yield: 89%).

MS: m/z=347 [M+H]⁺.

### e) N-[2-Chloro-6-(4-fluoro-phenyl)-5-(2-methylsulfanyl-pyrimidin-4-yl)-pyridin-3-yl]-acetamide

To a solution of 2-chloro-6-(4-fluoro-phenyl)-5-(2-methylsulfanyl-pyrimidin-4-yl)-pyridin-3-ylamine (0.19 g, 0.55 mmol, obtained in reference example 10d) in dichloromethane (6 mL), pyridine (0.22 mL, 2.74 mmol) and acetyl chloride (0.078 mL, 1.10 mmol) were subsequently added at 0° C. The mixture was stirred for 1 h. NaHCO₃ solution was added and extracted with dichloromethane (2×). The combined organic phases were washed with NaHCO₃ solution (2×), 2 M HCl solution (2×) and brine (1×), dried over Na₂SO₄ and concentrated to dryness to afford 0.20 g of the title compound (yield: 94%).

MS: m/z=389 [M+H]⁺.

### Example 1

### Methyl 6,7-bis(4-fluorophenyl)-6-(4-pyridyl)thieno[3,2-b]pyridin-3-carboxylate

To a solution of 1-(4-fluorophenyl)-2-(4-pyridyl)ethanone (0.30 g, 1.4 mmol, obtained in reference example 1) in 2-methoxyethanol (2 mL) under argon, 4-fluorobenzaldehyde (170 mg, 1.4 mmol), methyl 4-aminothiophen-3-carboxylate (240 mg, 1.5 mmol), 2-methoxyethanol (2 mL) and HCl (37%, 40 mg, 0.4 mmol) were added. The resulting mixture was heated at reflux overnight. It was allowed to cool and CHCl₃, MeOH (1 drop) and 1 N NaOH solution were added. The aqueous phase was extracted with CHCl₃ (×3). The combined organic extracts were dried over Na₂SO₄ and the solvent was evaporated. The crude product obtained was purified by chromatography on silica gel using hexane-EtOAc mixtures of increasing polarity as eluent, affording 0.52 g of the desired compound (83% yield).

LC-MS (method 1): t_{R}=8.66 min; m/z=459.1 [M+H]⁺.

Following a similar procedure to that described in example 1, but using in each case the appropriate starting compounds, the products shown in the following table were obtained:

| | | | LC-MS | | |
|---|---|---|---|---|---|
| | | Starting | | t_{R} | m/z |
| Example | Compound name | compounds | Method | (min) | [M + H]⁺ |
| 2 | Methyl 4,6-bis(4- | Reference example 1, | 1 | 8.75 | 443.0 |
| | fluorophenyl)-5-(4- | methyl 5-aminofuran-2- | | | |
| | pyridyl)furo[2,3-b]pyridine- | carboxylate and 4- | | | |
| | 2-carboxylate | fluorobenzaldehyde | | | |
| 3 | Methyl 5,7-bis(4- | Reference example 1, | 1 | 7.83 | 456.1 |
| | fluorophenyl)-1-methyl-6- | methyl 4-amino-1- | | | |
| | (4-pyridyl)pyrrolo[3,2- | methylpyrrole-2-carboxylate | | | |
| | b]pyridine-2-carboxylate | and 4-fluorobenzaldehyde | | | |
| 4 | 4,6-Bis(4-fluorophenyl)-3- | Reference example 1,5- | 1 | 8.36 | 400.1 |
| | methyl-5-(4- | amino-3-(methyl)isoxazole | | | |
| | pyridyl)isoxazolo[5,4- | and 4-fluorobenzaldehyde | | | |
| | b]pyridine | | | | |
| 5 | Ethyl 5,7-bis(4- | Reference example 1, ethyl | 1 | 7.01 | 471.2 |
| | fluorophenyl)-1-methyl-6- | 4-amino-1-methylimidazole- | | | |
| | (4-pyridyl)imidazo[4,5- | 2-carboxylate and 4- | | | |
| | b]pyridine-2-carboxylate | fluorobenzaldehyde | | | |

### Example 6

### [5,7-Bis(4-fluorophenyl)-1-methyl-6-(4-pyridyl)pyrrolo[3,2-b]pyridin-2-yl]methanol

A suspension of CaCl₂ (73 mg, 0.7 mmol) and NaBH₄ (50 mg, 1.3 mmol) in THF (16 mL) under argon was heated at reflux for 4 h. It was cooled to 30° C. and a solution of methyl 5,7-bis(4-fluorophenyl)-1-methyl-6-(4-pyridyl)pyrrolo[3,2-b]pyridine-2-carboxylate (100 mg, 0.2 mmol, obtained in example 3) in THF (24 mL) was added dropwise. The resulting mixture was heated at reflux for 6 h. It was allowed to cool, it was poured into ice and THF was evaporated. The residue was extracted twice with CH₂Cl₂. The combined organic extracts were dried over Na₂SO₄ and the solvent was evaporated. The crude product obtained was purified by chromatography on silica gel using hexane-EtOAc mixtures of increasing polarity as eluent, affording 25 mg of the desired compound (26% yield).

LC-MS (method 1): t_{R}=4.41 min; m/z=428.1 [M+H]⁺.

### Example 7

### [5,7-Bis(4-fluorophenyl)-1-methyl-6-(4-pyridyl)imidazo[4,5-b]pyridin-2-yl]methanol

Following a similar procedure to that described in example 6, but starting from ethyl 5,7-bis(4-fluorophenyl)-1-methyl-6-(4-pyridyl)imidazo[4,5-b]pyridine-2-carboxylate (obtained in example 5), the title compound was obtained.

LC-MS (method 1): t_{R}=5.00 min; m/z=429.1 [M+H]⁺.

### Example 8

### 5,7-Bis(4-fluorophenyl)-2-methyl-6-(4-pyridyl)pyrazolo[1,5-a]pyrimidine

A solution of 3-amino-5-methyl-2H-pyrazole (70 mg, 0.7 mmol) in EtOH (2 mL) and 37% HCl (1 drop), was added under argon atmosphere over 1-(4-fluorophenyl)-2-(4-pyridyl)vinyl 4-fluorobenzoate (0.22 g, 65.0 mmol, obtained in reference example 3). The resulting mixture was heated at reflux overnight. It was allowed to cool and the solvent was evaporated. The crude product obtained was purified by chromatography on silica gel using hexane-EtOAc mixtures of increasing polarity as eluent, affording 9 mg of the title compound (3% yield).

LC-MS (method 1, flow 0.30 mL/min): t_{R}=8.04 min; m/z=399.1 [M+H]⁺.

### Example 9

### 2-Methyl-5,7-diphenyl-6-(4-pyridyl)pyrazolo[1,5-a]pyrimidine

Following a similar procedure to that described in example 8, but using 1-phenyl-2-(4-pyridyl)vinyl benzoate (obtained in reference example 4) instead of 1-(4-fluorophenyl)-2-(4-pyridyl)vinyl 4-fluorobenzoate, the title compound was obtained.

LC-MS (method 1, flow: 0.30 mL/min): t_{R}=6.72 min; m/z=363.2 [M+H]⁺.

### Example 10

### 5,7-Bis(4-fluorophenyl)-1-methyl-6-(4-pyridyl)imidazo[4,5-b]pyridine

### a) 5,7-Bis(4-fluorophenyl)-1-methyl-6-(4-pyridyl)imidazo[4,5-b]pyridine-2-carboxylic acid

To a solution of ethyl 5,7-bis(4-fluorophenyl)-1-methyl-6-(4-pyridyl)imidazo[4,5-b]pyridine-2-carboxylate (0.29 g, 0.6 mmol, obtained in example 5) in EtOH (13 mL) a solution of KOH (0.42 g, 6.3 mmol) in water (2.5 mL) was added and the resulting mixture was heated at reflux for 2 h. It was allowed to cool and the solvent was evaporated. Water was added and then the mixture was brought to pH 6-7 with 1 N HCl. It was extracted with EtOAc and the organic phase was dried over Na₂SO₄ and the solvent was evaporated. The crude product obtained was purified by chromatography on silica gel using EtOAc-MeOH—NH₃ Mixtures of increasing polarity as eluent, affording 253 mg of the desired compound (quantitative yield).

LC-MS (method 1): t_{R}=5.16 min; m/z=399.2 [M-CO₂+H]⁺.

### b) Title Compound

5,7-Bis(4-fluorophenyl)-1-methyl-6-(4-pyridyl)imidazo[4,5-b]pyridine-2-carboxylic acid (50 mg, 0.1 mmol, obtained in section a) was heated at 200° C. overnight. The crude product obtained was purified by chromatography on silica gel using EtOAc-MeOH mixtures of increasing polarity as eluent, affording 39 mg of the title compound (89% yield).

LC-MS (method 1): t_{R}=5.37 min; m/z=399.1 [M+H]⁺.

### Example 11

### 5,7-Bis(4-fluorophenyl)-N-(2-hydroxyethyl)-6-(4-pyridyl)thieno[3,2-b]pyridine-3-carboxamide

### a) 5,7-Bis(4-fluorophenyl)-6-(4-pyridyl)thieno[3,2-b]pyridine-3-carboxylic acid

Following a similar procedure to that described in section a of example 10, but using methyl 5,7-bis(4-fluorophenyl)-6-(4-pyridyl)thieno[3,2-b]pyridine-3-carboxylate as starting compound (obtained in example 1), the title compound was obtained.

LC-MS (method 1): t_{R}=8.22 min; m/z=445.1 [M+H]⁺.

### b) Title Compound

To a solution of 5,7-bis(4-fluorophenyl)-6-(4-pyridyl)thieno[3,2-b]pyridine-3-carboxylic acid (100 mg, 0.2 mmol, obtained in section a) in DMF (1.5 mL), 1-hydroxybenzotriazole (31 mg, 0.2 mmol), N-(3-dimethylaminopropyl)-N′-ethylcarbodiimide (53 mg, 0.3 mmol) and NMM (35 mg, 0.3 mmol) were added, and the resulting mixture was stirred at room temperature for 1 h. 2-Aminoethanol (14 mg, 0.2 mmol) was added and the mixture was stirred at room temperature overnight. It was poured into water and extracted with CHCl₃. The organic phase was dried over Na₂SO₄ and concentrated. The crude product obtained was purified by chromatography on silica gel using EtOAc-MeOH mixtures of increasing polarity as eluent, affording 41 mg of the title compound (40% yield).

LC-MS (method 1): t_{R}=6.74 min; m/z=488.1 [M+H]⁺.

### Example 12

### 5,7-Bis(4-fluorophenyl)-1-methyl-6-(4-pyridyl)pyrrolo[3,2-b]pyridine-2-carboxamide

### a) 5,7-Bis(4-fluorophenyl)-1-methyl-6-(4-pyridyl)pyrrolo[3,2-b]pyridine-2-carboxylic acid

Following a similar procedure to that described in section a of example 10, but using methyl 5,7-bis(4-fluorophenyl)-1-methyl-6-(4-pyridyl)pyrrolo[3,2-b]pyridine-2-carboxylate (obtained in example 3) as starting compound, the title compound was obtained.

LC-MS (method 1): t_{R}=5.32 min; m/z=442.1 [M+H]⁺.

### b) Title Compound

Following a similar procedure to that described in section b of example 11, but using 5,7-bis(4-fluorophenyl)-1-methyl-6-(4-pyridyl)pyrrolo[3,2-b]pyridine-2-carboxylic acid (obtained in section a) and ammonia as starting compounds, the title compound was obtained.

LC-MS (method 1): t_{R}=5.12 min; m/z=441.1 [M+H]⁺. Following a similar procedure to that described in section b of example 12, but using in each case the appropriate starting compounds, the products shown in the following table were obtained:

| | | | LC-MS | | |
|---|---|---|---|---|---|
| | | Starting | | t_{R} | m/z |
| Example | Compound name | compounds | Method | (min) | [M + H]⁺ |
| 13 | 5,7-Bis(4-fluorophenyl)-N-(2-hydroxyethyl)- | Example 12 | 1 | 4.86 | 485.1 |
| | 1-methyl-6-(4-pyridyl)pyrrolo[3,2- | section a and | | | |
| | b]pyridine-2-carboxamide | 2-aminoethanol | | | |
| 14 | [5,7-Bis(4-fluorophenyl)-1-methyl-6-(4- | Example 12 | 1 | 5.44 | 511.1 |
| | pyridyl)pyrrolo[3,2-b]pyridin-2-yl]morpholin- | section a and | | | |
| | 4-ylmethanone | morpholine | | | |

### Example 15

### 3-Amino-5,7-bis(4-fluorophenyl)-6-(4-pyridyl)thieno[3,2-b]pyridine

To a solution of 5,7-bis(4-fluorophenyl)-6-(4-pyridyl)thieno[3,2-b]pyridine-3-carboxylic acid (100 mg, 0.2 mmol, obtained in section a of example 11) in DMF (0.13 mL) under argon, a solution of TEA (35 mg, 0.3 mmol) in DMF (0.33 mL) was added and then a solution of diphenylphosphorylazide (95 mg, 0.3 mmol) in DMF (0.33 mL) was added dropwise. The resulting mixture was stirred at room temperature for 3 h. Water (2 mL) was slowly added and the mixture was heated at 100° C. for 1 h. It was allowed to cool to room temperature and the solvent was evaporated. The residue was diluted with CHCl₃ and washed with saturated NaHCO₃ solution (×3). The organic phase was dried over Na₂SO₄ and concentrated. The crude product obtained was purified by chromatography on silica gel using hexane-EtOAc mixtures of increasing polarity as eluent, affording 21 mg of the title compound (23% yield).

LC-MS (method 2): t_{R}=9.46 min; m/z=416.1 [M+H]⁺.

### Example 16

### 2-[4,6-Bis-(4-fluorophenyl)-5-(4-pyridyl)furo[2,3-b]pyridin-2-yl]propan-2-ol

To a solution of methyl 4,6-bis(4-fluorophenyl)-5-(4-pyridyl)furo[2,3-b]pyridine-2-carboxylate (200 mg, 0.4 mmol, obtained in example 2) in THF (0.7 mL) cooled to 0° C., a 3 M solution of methylmagnesium chloride in THF (0.60 mL, 1.8 mmol) was added under argon. The resulting mixture was stirred at room temperature for 2 h. EtOAc and saturated NH₄Cl solution were added and the phases were separated. The organic phase was dried over Na₂SO₄ and concentrated. The crude product obtained was purified by chromatography on silica gel using hexane-EtOAc mixtures of increasing polarity as eluent, affording 152 mg of the title compound (76% yield).

LC-MS (method 1): t_{R}=7.04 min; m/z=443.2 [M+H]+

Following a similar procedure to that described in example 16, but using the appropriate starting compounds in each case, the products shown in the following table were obtained:

| | | | LC-MS | | |
|---|---|---|---|---|---|
| | | Starting | | t_{R} | m/z |
| Example | Compound name | compound | Method | (min) | [M + H]⁺ |
| 17 | 2-[5,7-Bis(4-fluorophenyl)-6-(4- | Example 1 | 1 | 8.82 | 459.1 |
| | pyridyl)thieno[3,2-b]pyridin-3-yl]propan-2-ol | | | | |
| 18 | 2-[5,7-Bis(4-fluorophenyl)-1-methyl-6-(4- | Example 5 | 1 | 5.32 | 457.2 |
| | pyridyl)imidazo[4,5-b]pyridin-2-yl]propan-2-ol | | | | |
| 19 | 1-[5,7-Bis(4-fluorophenyl)-1-methyl-6-(4- | Example 5 | 1 | 6.66 | 441.1 |
| | pyridyl)imidazo[4,5-b]pyridin-2-yl]ethanone | | | | |
| 20 | 2-[5,7-Bis(4-fluorophenyl)-1-methyl-6-(4- | Example 3 | 1 | 5.17 | 456.2 |
| | pyridyl)pyrrolo[3,2-b]pyridin-2-yl]propan-2-ol | | | | |
| 21 | 1-[5,7-Bis(4-fluorophenyl)-1-methyl-6-(4- | Example 3 | 1 | 7.08 | 440.1 |
| | pyridyl)pyrrolo[3,2-b]pyridin-2-yl]ethanone | | | | |

### Example 22

### [4,6-Bis(4-fluorophenyl)-5-(4-pyridyl)furo[2,3-b]pyridin-2-yl]methanol

Following a similar procedure to that described in example 6, but using methyl 4,6-bis(4-fluorophenyl)-5-(4-pyridyl)furo[2,3-b]pyridine-2-carboxylate (obtained in example 2) as starting compound, the title compound was obtained.

LC-MS (method 1): t_{R}=6.26 min; m/z=415.0 [M+H]⁺.

### Example 23

### 4,6-Bis-(4-fluoro-phenyl)-5-pyridin-4-yl-furo[2,3-b]pyridine-2-carboxylic acid (2-methoxy-ethyl)-amide

### a) 4,6-Bis-(4-fluoro-phenyl)-5-pyridin-4-yl-furo[2,3-b]pyridine-2-carboxylic acid

Following a similar procedure to that described in example 10a, but starting from methyl 4,6-bis(4-fluorophenyl)-5-(4-pyridyl)furo[2,3-b]pyridine-2-carboxylate (obtained in example 2), the title compound was obtained (yield: 95%).

LC-MS (method 3): t_{R}=2.6 min; m/z=429 [M+H]⁺.

### b) 4,6-Bis-(4-fluoro-phenyl)-5-pyridin-4-yl-furo[2,3-b]pyridine-2-carbonyl chloride

To a solution of 4,6-bis-(4-fluoro-phenyl)-5-pyridin-4-yl-furo[2,3-b]pyridine-2-carboxylic acid (0.20 g, 0.47 mmol, obtained in example 23a) in 1,2-dichloropropane (4 mL), thionyl chloride (0.068 mL, 0.94 mmol) was added dropwise and under nitrogen atmosphere. The mixture was heated to reflux for 1 h under nitrogen atmosphere. It was allowed to cool and then concentrated. The residue was dissolved in toluene and concentrated to dryness, to afford the title compound (yield: 95%).

### c) 4,6-Bis-(4-fluoro-phenyl)-5-pyridin-4-yl-furo[2,3-b]pyridine-2-carboxylic acid (2-methoxy-ethyl)-amide

To a solution of 4,6-bis-(4-fluoro-phenyl)-5-pyridin-4-yl-furo[2,3-b]pyridine-2-carbonyl chloride (0.05 g, 0.11 mmol, obtained in example 23b) in CH₂Cl₂ (1 mL), 2-methoxyethylamine (0.05 g, 0.68 mmol) was added. The mixture was stirred overnight at room temperature. CH₂Cl₂ was added and washed with 3% citric acid aqueous solution (3×) and saturated NaHCO₃ (2×). The aqueous phase was extracted with CH₂Cl₂ (2×). The organic phase was dried over MgSO₄ and concentrated to dryness. The crude product obtained was purified by chromatography on silica gel using heptane/EtOAc mixtures of increasing polarity as eluent, to afford 47 mg of the desired product as a white solid (yield: 88%).

LC-MS (method 3): t_{R}−2.47 min; m/z=486 [M+H]⁺.

### Examples 24-26

Following a similar procedure to that described in example 23c, but using the appropriate amine in each case, the compounds in the following table were obtained:

| | | | LC-MS | | |
|---|---|---|---|---|---|
| Ex | Compound name | Amine | Method | t_{R} (min) | m/z [M + H]⁺ |
| 24 | 4,6-Bis-(4-fluoro-phenyl)-5-pyridin-4-yl- | 1-propylamine | 3 | 2.63 | 470 |
| | furo[2,3-b]pyridine-2-carboxylic acid | | | | |
| | propylamide | | | | |
| 25 | 4,6-Bis-(4-fluoro-phenyl)-5-pyridin-4-yl- | 2-morpholin-4- | 3 | 2.31 | 541 |
| | furo[2,3-b]pyridine-2-carboxylic acid (2- | ylethylamine | | | |
| | morpholin-4-yl-ethyl)-amide | | | | |
| 26 | 4,6-Bis-(4-fluoro-phenyl)-5-pyridin-4-yl- | 2-piperidin-1- | 3 | 2.41 | 539 |
| | furo[2,3-b]pyridine-2-carboxylic acid (2- | ylethylamine | | | |
| | piperidin-1-yl-ethyl)-amide | | | | |

### Example 27

### 4,6-Bis-(4-fluoro-phenyl)-5-pyridin-4-yl-furo[2,3-b]pyridine-2-carboxylic acid (2-hydroxy-ethyl)-amide

To a solution of 4,6-Bis-(4-fluoro-phenyl)-5-pyridin-4-yl-furo[2,3-b]pyridine-2-carboxylic acid (0.058 g, 0.14 mmol, obtained in example 23a) and TEA (0.077 mL, 0.56 mmol) in CH₂Cl₂ (2 mL), 2-aminoethanol (41 mg, 0.68 mmol) and 1,3-dimethylimidazolidiniumhexafluorophosphate (163 mg, 0.68 mmol) were added. The mixture was heated under microwave irradiation at 110° C. for 20 min. After cooling down, CH₂Cl₂ was added and the mixture was washed with 0.5 N HCl aqueous solution (3×). The aqueous phase was extracted with CH₂Cl₂ (2×). The organic phase was dried over MgSO₄ and concentrated to dryness. The crude product obtained was purified by chromatography on silica gel using CH₂Cl₂/MeOH mixtures of increasing polarity as eluent, to afford 5 mg of the desired product as a white solid (yield: 8%).

LC-MS (method 3): t_{R}=2.57 min; m/z=472 [M+H]⁺.

### Examples 28-31

Following a similar procedure to that described in example 23, but starting from example 3 instead of from example 2 and using the appropriate amine in step c) in each case, the compounds in the following table were obtained:

| | | | HPLC | | MS |
|---|---|---|---|---|---|
| Ex | Compound name | Amine | Method | t_{R} (min) | m/z [M + H]⁺ |
| 28 | 5,7-Bis-(4-fluoro-phenyl)-1-methyl-6- | 2- | 5 | 10.76 | 499 |
| | pyridin-4-yl-1H-pyrrolo[3,2- | methoxyethylamine | | | |
| | b]pyridine-2-carboxylic acid (2- | | | | |
| | methoxy-ethyl)-amide | | | | |
| 29 | 5,7-Bis-(4-fluoro-phenyl)-1-methyl-6- | 1-propylamine | 5 | 12.3 | 483 |
| | pyridin-4-yl-1H-pyrrolo[3,2- | | | | |
| | b]pyridine-2-carboxylic acid | | | | |
| | propylamide | | | | |
| 30 | 5,7-Bis-(4-fluoro-phenyl)-1-methyl-6- | 2-morpholin-4- | 5 | 8.29 | 554 |
| | pyridin-4-yl-1H-pyrrolo[3,2- | ylethylamine | | | |
| | b]pyridine-2-carboxylic acid (2- | | | | |
| | morpholin-4-yl-ethyl)-amide | | | | |
| 31 | 5,7-Bis-(4-fluoro-phenyl)-1-methyl-6- | 2-piperidin-1- | 5 | 9.38 | 552 |
| | pyridin-4-yl-1H-pyrrolo[3,2- | ylethylamine | | | |
| | b]pyridine-2-carboxylic acid (2- | | | | |
| | piperidin-1-yl-ethyl)-amide | | | | |

### Example 32

### [5,7-Bis-(4-fluoro-phenyl)-1-methyl-6-pyridin-4-yl-1H-pyrrolo[3,2-b]pyridin-2-ylmethyl]-(2-methoxy-ethyl)-amine

### a) 5,7-Bis-(4-fluoro-phenyl)-1-methyl-6-pyridin-4-yl-1H-pyrrolo[3,2-b]pyridine-2-carbaldehyde

To a solution of [5,7-bis(4-fluorophenyl)-1-methyl-6-(4-pyridyl)pyrrolo[3,2-b]pyridin-2-yl]methanol (0.445 g, 1.04 mmol, obtained in example 6) and TEA (0.725 mL, 5.2 mmol) in DMSO (3 mL), pyridine-SO₃ complex (0.496 g, 3.12 mmol) was added under nitrogen atmosphere. The mixture was stirred at room temperature for 1 h. It was then poured into ice and EtOAc was added. The organic phase was washed with water (2×). The aqueous phase was extracted with EtOAc (2×). The organic phase was dried over MgSO₄ and concentrated to dryness, to afford 395 mg of the desired product as a white solid (yield: 90%).

LC-MS (method 3): t_{R}=2.63 min; m/z=426 [M+H]⁺.

### b) [5,7-Bis-(4-fluoro-phenyl)-1-methyl-6-pyridin-4-yl-1H-pyrrolo[3,2-b]pyridin-2-ylmethyl]-(2-methoxy-ethyl)-amine

To a solution of 5,7-bis-(4-fluoro-phenyl)-1-methyl-6-pyridin-4-yl-1H-pyrrolo[3,2-b]pyridine-2-carbaldehyde (0.099 g, 0.23 mmol, obtained in example 32a) in CH₂Cl₂ (1 mL), 2-methoxyethylamine (0.10 mL, 1.15 mmol) was added at room temperature. The pH of the mixture was adjusted to pH=6 with acetic acid and it was stirred for 2 h at room temperature. Then, Na(OAc)₃BH (0.244 g, 1.15 mmol) was added and the reaction was stirred at room temperature overnight. Saturated NaHCO₃ aqueous solution and EtOAc were added. The organic phase was washed with saturated Na₂CO₃ aqueous solution (2×). The aqueous phase was extracted with EtOAc (2×). The organic phase was dried over MgSO₄ and concentrated to dryness. The crude product obtained was purified by chromatography on silica gel using CH₂Cl₂/MeOH mixtures of increasing polarity as eluent, to afford 49 mg of the desired product as a white solid (yield: 44%).

LC-MS (method 3): t_{R}=2.41 min; m/z=485 [M+H]⁺.

### Example 33

### [5,7-Bis-(4-fluoro-phenyl)-1-methyl-6-pyridin-4-yl-1H-pyrrolo[3,2-b]pyridin-2-ylmethyl]-cyclopropylmethyl-amine

Following a similar procedure to that described in example 32b, but using c-cyclopropyl-methylamine instead of 2-methoxyethylamine, the title compound was obtained as a white solid (58 mg, yield: 52%).

LC-MS (method 3): t_{R}=2.40 min; m/z=481 [M+H]⁺.

### Examples 34 nd 35

### {[5,7-Bis-(4-fluoro-phenyl)-1-methyl-6-pyridin-4-yl-1H-pyrrolo[3,2-b]pyridin-2-ylmethyl]-amino}-acetic acid methyl ester (34)

### {[5,7-Bis-(4-fluoro-phenyl)-1-methyl-6-pyridin-4-yl-1H-pyrrolo[3,2-b]pyridin-2-ylmethyl]-N-ethyl-amino}-acetic acid methyl ester (35)

Following a similar procedure to that described in example 32b, but using amino-acetic acid methyl ester instead of 2-methoxyethylamine, the title compounds were obtained as white solids.

Example 34

9 mg, yield: 8%

LC-MS (method 3): t_{R}=2.39 min; m/z=499 [M+H]⁺.

Example 35

8 mg, yield: 7%.

LC-MS (method 3): t_{R}=2.45 min; m/z=527 [M+H]⁺.

### Example 36

### [567-Bis-(4-fluoro-phenyl)-1-methyl-6-pyridin-4-yl-1H-pyrrolo[3,2-b]pyridin-2-ylmethyl]-propyl-amine

Following a similar procedure to that described in example 32b, but using 1-propylamine instead of 2-methoxyethylamine, the title compound was obtained as a white solid (6 mg, yield: 29%).

LC-MS (method 3): t_{R}=2.41 min; m/z=469 [M+H]⁺.

### Example 37

### 5,7-Bis-(4-fluoro-phenyl)-1-methyl-6-pyridin-4-yl-1H-pyrrolo[3,2-b]pyridine

To a solution of [5,7-bis(4-fluorophenyl)-1-methyl-6-(4-pyridyl)pyrrolo[3,2-b]pyridin-2-yl]methanol (0.05 g, 0.11 mmol, obtained in example 6) in dioxane (1 mL), KOtBu (0.025 g, 0.22 mmol) and 4-(2-chloro-ethyl)-morpholine. HCl (0.020 mg, 0.11 mmol) was added and the reaction was stirred at room temperature overnight. It was acidified with HCl aqueous solution to pH=7 and then EtOAc was added. The organic phase was washed with saturated Na₂CO₃ aqueous solution (3×). The aqueous phase was extracted with EtOAc (2×). The organic phase was dried over MgSO₄ and concentrated to dryness. The crude product obtained was purified by chromatography on silica gel using CH₂Cl₂/MeOH mixtures of increasing polarity as eluent, to afford 8 mg of the desired product as a white solid (yield: 19%).

LC-MS (method 3): t_{R}=2.29 min; m/z=398 [M+H]⁺.

### Example 38

### [5,7-Bis-(4-fluoro-phenyl)-1-methyl-6-pyridin-4-yl-1H-imidazo[4,5-b]pyridin-2-yl]-morpholin-4-yl-methanone

To a solution of ethyl 5,7-bis(4-fluorophenyl)-1-methyl-6-(4-pyridyl)imidazo[4,5-b]pyridine-2-carboxylate (60 mg, 0.13 mmol, obtained in example 5) in EtOH (2 mL) was added morpholine (330 μL, 3.83 mmol). The resulting mixture was heated to 150° C. for 20 min using microwave irradiation. After evaporation of the solvent, the crude product was purified by preparative HPLC and lyophilized, to afford the title compound as a white solid (yield: 20%).

HPLC (method 6): t_{R}=10.87 min. MS: m/z=512 [M+H]⁺.

### Example 39

### 5,7-Bis-(4-fluoro-phenyl)-6-pyridin-4-yl-1H-pyrazolo[4,3-b]pyridine-3-carboxylic acid methyl ester

Following a similar procedure to that described in example 1, but using 4-amino-1H-pyrazole-3-carboxylic acid methyl ester (obtained in reference example 7) instead of methyl 4-aminothiophen-3-carboxylate, and ethanol as a solvent, 5 mg of the title compound were obtained as a white solid (yield: 5%).

HPLC (method 4): t_{R}=6.17 min. MS: m/z=443 [M+H]⁺.

### Example 40

### Cyclopropylmethyl-{4-[6-(4-fluoro-phenyl)-3-methyl-isoxazolo[5,4-b]pyridin-5-yl]-pyrimidin-2-yl}-amine

### a) 6-(4-Fluoro-phenyl)-3-methyl-5-(2-methylsulfanyl-pyrimidin-4-yl)-isoxazolo[5,4-b]pyridine

To a solution of 1-(4-fluoro-phenyl)-2-(2-methylsulfanyl-pyrimidin-4-yl)-propenone (1.08 g, 3.93 mmol, obtained in reference example 5c) and 3-methyl-isoxazol-5-ylamine (0.42 g, 4.32 mmol) in EtOH (30 mL), 37% HCl aqueous solution (0.113 mL, 1.18 mmol) was added. The reaction was stirred for 2 days at room temperature. Next, cerium (IV) ammonium nitrate was added in order to complete the reaction. The reaction mixture was washed with saturated NaHCO₃ aqueous solution (3×). The aqueous phase was extracted with EtOAc. The organic phase was dried over MgSO₄ and concentrated to dryness. The crude product obtained was purified by chromatography on silica gel using heptane/EtOAc mixtures of increasing polarity as eluent, to afford 523 mg of the desired product as a white solid (yield: 38%).

LC-MS (method 3): t_{R}=3.03 min; m/z=353 [M+H]⁺.

### b) 6-(4-Fluoro-phenyl)-5-(2-methanesulfonyl-pyrimidin-4-yl)-3-methyl-isoxazolo[5,4-b]pyridine

To a solution of 6-(4-fluoro-phenyl)-3-methyl-5-(2-methylsulfanyl-pyrimidin-4-yl)-isoxazolo[5,4-b]pyridine (0.1 g, 0.28 mmol) in MeOH (5 mL), Oxone® (0.87 g, 1.42 mmol) in water (5 mL) was added. The mixture was stirred for 1 h at room temperature. After evaporation of methanol, EtOAc and saturated NaHCO₃ aqueous solution was added. The organic phase was washed with saturated NaHCO₃ aqueous solution (2×). The aqueous phase was extracted with EtOAc (2×). The organic phase was dried over MgSO₄ and concentrated to dryness. The crude product obtained was purified by chromatography on silica gel using CH₂Cl₂/MeOH mixtures of increasing polarity as eluent, to afford 47 mg of the desired product as a white solid (yield: 56%).

LC-MS (method 3): t_{R}=2.82 min; m/z=385 [M+H]⁺.

### c) Cyclopropylmethyl-{4-[6-(4-fluoro-phenyl)-3-methyl-isoxazolo[5,4-b]pyridin-5-yl]-pyrimidin-2-yl}-amine

To a solution of 6-(4-fluoro-phenyl)-5-(2-methanesulfonyl-pyrimidin-4-yl)-3-methyl-isoxazolo[5,4-b]pyridine (0.045 g, 0.12 mmol) in THF (0.5 mL), C-cyclopropyl-methylamine (0.052 mL, 0.60 mmol) was added. The reaction was heated at 50° C. for 2.5 h. The organic phase was washed with water and brine (2×). The aqueous phase was extracted with EtOAc. The organic phase was dried over MgSO₄ and concentrated to dryness. The crude product obtained was purified by chromatography on silica gel using heptane/EtOAc mixtures of increasing polarity as eluent, to afford 41 mg of the desired product as a white solid (yield: 91%).

LC-MS (method 3): t_{R}=2.82 min; m/z=376 [M+H]⁺.

### Examples 41-42

Following a similar procedure to that described in example 40c, but using the appropriate amine in each case, the compounds in the following table were obtained:

| | | | LC-MS | | |
|---|---|---|---|---|---|
| Ex | Compound name | Amine | Method | t_{R} (min) | m/z [M + H]⁺ |
| 41 | {4-[6-(4-Fluoro-phenyl)-3-methyl- | 3-methoxy- | 3 | 2.72 | 394 |
| | isoxazolo[5,4-b]pyridin-5-yl]-pyrimidin- | propylamine | | | |
| | 2-yl}-(3-methoxy-propyl)-amine | | | | |
| 42 | (S)-{4-[6-(4-Fluoro-phenyl)-3-methyl- | (S)-1-phenyl- | 3 | 3.01 | 426 |
| | isoxazolo[5,4-b]pyridin-5-yl]-pyrimidin- | ethylamine | | | |
| | 2-yl}-(1-phenyl-ethyl)-amine | | | | |

### Example 43

### Cyclopropylmethyl-{4-[6-(4-fluoro-phenyl)-3-methyl-isothiazolo[5,4-b]pyridin-5-yl]-pyrimidin-2-yl}-amine

### a) 6-(4-Fluoro-phenyl)-3-methyl-5-(2-methylsulfanyl-pyrimidin-4-yl)-isothiazolo[5,4-b]pyridine

Following a similar procedure to that described in example 40a, but using 3-methyl-isothiazol-5-ylamine instead of 3-methyl-isoxazol-5-ylamine, the title compound was obtained as a white solid (202 mg, yield: 31%).

LC-MS (method 3): t_{R}=2.96 min; m/z=369 [M+H]⁺.

### b) 6-(4-Fluoro-phenyl)-5-(2-methanesulfonyl-pyrimidin-4-yl)-3-methyl-isothiazolo[5,4-b]pyridine

Following a similar procedure to that described in example 40b, but using 6-(4-fluoro-phenyl)-3-methyl-5-(2-methylsulfanyl-pyrimidin-4-yl)-isothiazolo[5,4-b]pyridine instead of 6-(4-fluoro-phenyl)-3-methyl-5-(2-methylsulfanyl-pyrimidin-4-yl)-isoxazolo[5,4-b]pyridine, the title compound was obtained as a white solid (204 mg, yield: 93%).

MS: m/z=401 [M+H]⁺.

### c) Cyclopropylmethyl-{4-[6-(4-fluoro-phenyl)-3-methyl-isothiazolo[5,4-b]pyridin-5-yl]-pyrimidin-2-yl}-amine

Following a similar procedure to that described in example 40c, but using 6-(4-fluoro-phenyl)-5-(2-methanesulfonyl-pyrimidin-4-yl)-3-methyl-isothiazolo[5,4-b]pyridine instead of 6-(4-fluoro-phenyl)-5-(2-methanesulfonyl-pyrimidin-4-yl)-3-methyl-isoxazolo[5,4-b]pyridine, the title compound was obtained as a white solid (44 mg, yield: 66%).

LC-MS (method 3): t_{R}=2.90 min; m/z=392 [M+H]⁺.

### Examples 44-45

Following a similar procedure to that described in example 43, but using the appropriate amine in step c) in each case, the compounds in the following table were obtained:

| | | | LC-MS | | |
|---|---|---|---|---|---|
| Ex | Compound name | Amine | Method | t_{R} (min) | m/z [M + H]⁺ |
| 44 | {4-[6-(4-Fluoro-phenyl)-3-methyl- | 3-methoxy- | 3 | 2.79 | 410 |
| | isothiazolo[5,4-b]pyridin-5-yl]-pyrimidin- | propylamine | | | |
| | 2-yl}-(3-methoxy-propyl)-amine | | | | |
| 45 | (S)-{4-[6-(4-Fluoro-phenyl)-3-methyl- | (S)-1-phenyl- | 3 | 3.11 | 442 |
| | isothiazolo[5,4-b]pyridin-5-yl]-pyrimidin- | ethylamine | | | |
| | 2-yl}-(1-phenyl-ethyl)-amine | | | | |

### Example 46

### Cyclopropylmethyl-{4-[5-(4-methoxy-phenyl)-1H-pyrrolo[3,2-b]pyridin-6-yl]-pyrimidin-2-yl}-amine

### a) 5-(4-Methoxy-phenyl)-6-(2-methylsulfanyl-pyrimidin-4-yl)-1H-pyrrolo[3,2-b]pyridine

Following a similar procedure to that described in example 40a, but using 1H-pyrrol-3-ylamine instead of 3-methyl-isoxazol-5-ylamine, and reference example 6 instead of reference example 5, the title compound was obtained as a white solid (581 mg, yield: 86%)

MS: m/z=385.2 [M+H]⁺.

### b) 6-(2-Methanesulfonyl-pyrimidin-4-yl)-5-(4-methoxy-phenyl)-1H-pyrrolo[3,2-b]pyridine

Following a similar procedure to that described in example 40b, but using 5-(4-methoxy-phenyl)-6-(2-methylsulfanyl-pyrimidin-4-yl)-1H-pyrrolo[3,2-b]pyridine instead of 6-(4-fluoro-phenyl)-3-methyl-5-(2-methylsulfanyl-pyrimidin-4-yl)-isoxazolo[5,4-b]pyridine, the title compound was obtained as a white solid (154 mg, yield: 49%).

MS: m/z=417.2 [M+H]⁺.

### c) Cyclopropylmethyl-{4-[5-(4-methoxy-phenyl)-1H-pyrrolo[3,2-b]pyridin-6-yl]-pyrimidin-2-yl}-amine

Following a similar procedure to that described in example 40c, but using 6-(2-methanesulfonyl-pyrimidin-4-yl)-5-(4-methoxy-phenyl)-1H-pyrrolo[3,2-b]pyridine instead of 6-(4-fluoro-phenyl)-5-(2-methanesulfonyl-pyrimidin-4-yl)-3-methyl-isoxazolo[5,4-b]pyridine, the title compound was obtained as a white solid (4.5 mg, yield: 25%).

LC-MS (method 3): t_{R}=2.37 min; m/z=372 [M+H]⁺.

### Example 47

### (S)-{4-[5-(4-Methoxy-phenyl)-1H-pyrrolo[3,2-b]pyridin-6-yl]-pyrimidin-2-yl}-(1-phenyl-ethyl)-amine

Following a similar procedure to that described in example 46, but using (S)-1-phenyl-ethylamine instead of C-cyclopropylmethylamine, the title compound was obtained as a white solid (2 mg, yield: 12%).

LC-MS (method 3): t_{R}=2.56 min; m/z=422.2 [M+H]⁺.

### Example 48

### 6-(4-Fluoro-phenyl)-4-(2-fluoro-phenyl)-3-methyl-5-pyridin-4-yl-isoxazolo[5,4-b]pyridine

A solution of 1-(4-fluoro-phenyl)-2-pyridin-4-yl-ethanone (250 mg, 1.16 mmol), 2-fluorobenzaldehyde (125 μL, 1.16 mmol) and 3-methylisoxazole-5-amine (125 mg, 1.28 mmol) in EtOH was stirred at 45° C. for 65 h. After cooling down to room temperature water and cerium (IV) ammonium nitrate (636 mg, 1.16 mmol) were added and the reaction mixture was further stirred for 1 h. It was diluted with EtOAc and washed with saturated aqueous NaHCO₃ solution. The organic solvent was removed in vacuo, and the residue was purified by chromatography on silica gel using heptane/EtOAc mixtures of increasing polarity as eluent, to afford 264 mg of the desired product as a yellow solid (yield: 57%).

HPLC (method 5): t_{R}=15.81 min. MS: m/z=400 [M+H]⁺.

### Example 49

### 4,6-Bis-(4-fluoro-phenyl)-3-methyl-5-pyridin-4-yl-isothiazolo[5,4-b]pyridine

Following a similar procedure to that described in example 48, but using 4-fluorobenzaldehyde instead of 2-fluorobenzaldehyde, and using 5-amino-3-methylisothiazole hydrochloride instead of 3-methylisoxazole-5-amine, 139 mg of the title compound were obtained as a pale yellow solid (yield: 29%).

HPLC (method 5): t_{R}=16.34 min. MS: m/z=416 [M+H]⁺.

### Example 50

### 4-(2-Fluoro-phenyl)-6-(4-fluoro-phenyl)-3-methyl-5-pyridin-4-yl-isothiazolo[5,4-b]pyridine

Following a similar procedure to that described in example 48, but using 5-amino-3-methylisothiazole hydrochloride instead of 3-methylisoxazole-5-amine, 57 mg of the title compound were obtained as a pale yellow solid (yield: 12%).

HPLC (method 5): t_{R}=16.81 min. MS: m/z=416 [M+H]⁺.

### Example 51

### 3-Methyl-5-pyridin-4-yl-6-(3-trifluoromethyl-phenyl)-isoxazolo[3,4-b]pyridine

To a solution of 2-pyridin-4-yl-1-(3-trifluoromethyl-phenyl)-ethanone (50 mg, 0.2 mmol, obtained in reference example 9b) and (4-formyl-5-methyl-isoxazol-3-yl)-carbamic acid tert-butyl ester (106 mg, 0.47 mmol, obtained in reference example 8b) in EtOH (1 mL), piperidine (5 μL) and acetic acid (5 μL) were added. The reaction mixture was heated under microwave irradiation at 155° C. for 30 min. More piperidine (10 μL) and acetic acid (10 μL) were added and the reaction was heated again for 30 min at 155° C. It was then poured into water and EtOAc. The organic layer was dried over Na₂SO₄ and concentrated to dryness. The residue was purified by chromatography on silica gel using heptane-EtOAc mixtures of increasing polarity as eluent, to afford 4 mg of the desired compound (yield: 6%).

HPLC (method 5): t_{R}=13.37 min. MS: m/z=356 [M+H]⁺.

### Example 52

### Cyclopropylmethyl-{4-[5-(4-fluoro-phenyl)-2-methyl-thiazolo[5,4-b]pyridin-6-yl]-pyrimidin-2-yl}-amine

### a) 5-(4-Fluoro-phenyl)-2-methyl-6-(2-methylsulfanyl-pyrimidin-4-yl)-thiazolo[5,4-b]pyridine

To a solution of N-[2-chloro-6-(4-fluoro-phenyl)-5-(2-methylsulfanyl-pyrimidin-4-yl)-pyridin-3-yl]-acetamide (0.15 g, 0.38 mmol, obtained in reference example 10e) in pyridine (1.5 mL), phosphorus pentasulfide (0.22 g, 0.99 mmol) was added at room temperature and under nitrogen atmosphere. The mixture was heated to 120° C. and stirred for 2 h. It was then cooled to room temperature and water was added. The aqueous phase was extracted with dichloromethane (2×) and the combined organic phases were washed with 2M HCl solution (2×) and brine (1×), dried over Na₂SO₄ and concentrated to dryness. The crude product was purified by chromatography on silica gel using heptane/EtOAc mixtures of increasing polarity as eluent, to afford 64 mg of the title compound (yield: 45%).

MS: m/z=369 [M+H]⁺.

### b) 5-(4-Fluoro-phenyl)-6-(2-methanesulfonyl-pyrimidin-4-yl)-2-methyl-thiazolo[5,4-b]pyridine

Following a similar procedure to that described in example 40b, but using 5-(4-fluoro-phenyl)-2-methyl-6-(2-methylsulfanyl-pyrimidin-4-yl)-thiazolo[5,4-b]pyridine instead of 6-(4-fluoro-phenyl)-3-methyl-5-(2-methylsulfanyl-pyrimidin-4-yl)-isoxazolo[5,4-b]pyridine, the title compound was obtained as a white solid (52 mg, yield: 75%).

MS: m/z=401 [M+H]⁺.

### c) Cyclopropylmethyl-{4-[5-(4-fluoro-phenyl)-2-methyl-thiazolo[5,4-b]pyridin-6-yl]-pyrimidin-2-yl}-amine

Following a similar procedure to that described in example 40c, but using 5-(4-fluoro-phenyl)-6-(2-methanesulfonyl-pyrimidin-4-yl)-2-methyl-thiazolo[5,4-b]pyridine instead of 6-(4-fluoro-phenyl)-5-(2-methanesulfonyl-pyrimidin-4-yl)-3-methyl-isoxazolo[5,4-b]pyridine, the title compound was obtained in solid white form (10 mg, yield: 20%).

HPLC (method 5): t_{R}=17.41 min. MS: m/z=392 [M+H]⁺.

### Example 53

### 5,7-Bis-(4-fluoro-phenyl)-6-pyridin-4-yl-1H-pyrazolo[4,3-b]pyridine

To a solution of 5,7-bis-(4-fluoro-phenyl)-6-pyridin-4-yl-1H-pyrazolo[4,3-b]pyridine-3-carboxylic acid methyl ester (100 mg, 0.23 mmol, obtained in example 39) in NMP (1 mL), 2 N HCl (50 μl) was added. The resulting mixture was heated at 225° C. for 20 min under microwave irradiation. The reaction was poured into water and extracted with EtOAc. The organic layer was dried over Na₂SO₄ and concentrated. The residue was purified by preparative HPLC to afford 16 mg of the title compound as an off-white solid (yield: 18%).

HPLC (method 5): t_{R}=11.25 min. MS: m/z=385 [M+H]⁺.

### Example 54

### 5,7-Bis-(4-fluoro-phenyl)-6-pyridin-4-yl-1H-pyrazolo[4,3-b]pyridine-3-carboxylic acid (2-hydroxy-ethyl)-amide

A solution of 5,7-bis-(4-fluoro-phenyl)-6-pyridin-4-yl-1H-pyrazolo[4,3-b]pyridine-3-carboxylic acid methyl ester (100 mg, 0.23 mmol, obtained in example 39) in 2-aminoethanol (1 mL) was heated at 150° C. for 30 min under microwave irradiation. The reaction was poured into water and extracted with EtOAc. The organic layer was dried over Na₂SO₄ and concentrated in vacuo. The residue was purified by preparative HPLC to afford 42 mg of the title compound as an off-white solid (yield: 40%).

HPLC (method 5): t_{R}=9.30 min. MS: m/z=472 [M+H]⁺.

### Example 55

### 6-(4-Fluoro-phenyl)-3-methyl-5-pyridin-4-yl-isoxazolo[3,4-b]pyridine

Following a similar procedure to that described in example 51, but using 1-(4-fluorophenyl)-2-(4-pyridyl)ethanone (obtained in reference example 1) instead of 2-pyridin-4-yl-1-(3-trifluoromethyl-phenyl)-ethanone (obtained in reference example 9b), the title compound was obtained as a white solid (11 mg, yield: 5%).

HPLC (method 5): t_{R}=9.91 min. MS: m/z=306 [M+H]⁺.

### Example 56

### (S)-{4-[5-(4-Fluoro-phenyl)-2-methyl-thiazolo[5,4-b]pyridin-6-yl]-pyrimidin-2-yl}-(1-phenyl-ethyl)-amine

Following a similar procedure to that described in example 52c, but using (S)-1-phenyl-ethylamine instead of C-cyclopropylmethylamine, the title compound was obtained as a white solid (3 mg, yield: 6%).

HPLC (method 5): t_{R}=20.46 min. MS: m/z=442 [M+H]⁺.

### Example 57

### Biological Assays

Inhibition of p38α Enzyme Activity:

Compound stocks in 100% DMSO are first diluted in DMSO to a concentration of 1×10⁻³ up to 3.2×10⁻⁸ M and then further diluted in kinase assay buffer (10 mM Tris-HCl, pH 7.2, 10 mM MgCl₂, 0.01% tween 20, 0.05% NaN₃, 1 mM dithiothreitol) to a concentration range of 4×10⁻⁵ up to 1.3×10⁻⁹ M. Of each compound solution 5 μL is transferred into a 384-wells black Optiplate (Packard, 6007279), followed by the addition of 5 μL of ATP (Boehringer, 519987), 5 μL of Fluorescein-labeled EGFR (Epidermal Growth Factor Receptor) peptide substrate and 5 μL of active p38 cc kinase (GST-tagged fusion protein corresponding to full-length human p38α; expressed in E. coli by Upstate, 14-251), all diluted in kinase assay buffer (see final concentrations in Table 1). The mixture is incubated for 2 h at room temperature (RT). The reaction is stopped by the addition of 60 μL of IMAP binding reagent, which has been diluted 400-fold in IMAP binding buffer (stock concentration 5 times diluted in Milli Q). After incubation for 30 min at RT, FP is measured on an Analyst™ multimode fluorescence plate reader (Molecular Devices) at excitation wavelength of 485 nm and emission wavelength of 530 nm (1 sec/well).

TABLE 1
assay conditions

| Kinase | Final | | Final | ATP final |
|---|---|---|---|---|
| (from Upstate) | concentration | Substrate | concentration | concentration |
| p38α/SAPK2a, | 0.30 U/mL | LVEPLTPSGEAPNQK-(FI) | 240 nM | 20 μM |
| active | | | | |

Data handling is performed as follows: percentage effects are calculated based on no-p38-enzyme-addition as the maximum inhibitory effect and with p38 enzyme addition as the minimum inhibitory effect. In each experiment, individual compound concentrations are tested in duplicate and percentage effect is calculated for each concentration.

Compounds of all examples exhibited more than 50% inhibition at 10 μM in the above assay. Compounds of examples 1, 2, 3, 5, 6, 7, 10, 12, 13, 14, 16, 18, 19, 20, 21, 22, 25, 26, 27, 28, 29, 30, 31, 32, 33, 34, 35, 36, 37, 39, 40, 41, 42, 43, 44, 45, 46, 47, 52, 53, 54 and 56 exhibited more than 50% inhibition at 1 μM in the above assay.

1. A compound of general formula I 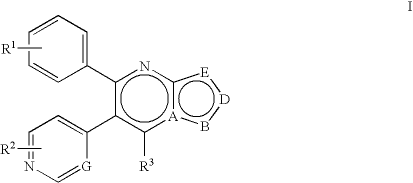
wherein:
A represents C or N;
B, D and E independently represent CR⁴, NR⁵, N, O or S;
with the following provisos:
a) when one of B, D or E represents O or S, the other two cannot represent O or S;
b) when A represents N, none of B, D, E can represent O or
S; and
c) when A represents C, B represents CR⁴ and one of D or E represents N or NR⁵, then the other of D or E cannot represent NR⁵ or N;
G represents N or C;
R¹ represents one or more substituents selected from H, R^{a}, halogen, —CN, —OH and —OR^{a};
R² represents one or more substituents selected from H, halogen and C₁₋₆alkyl, and additionally one substituent R² can also represent —OR^{b′}, —NO₂, —CN, —COR^{b′}, —CO₂R^{b′}, —CONR^{b′}R^{b′}, —NR^{b′}R^{b′}, —NR^{b′}COR^{b′}, —NR^{b′}CONR^{b′}R^{b′}, —NR^{b′}CO₂R^{b}, —NR^{b′}SO₂R^{b}, —SR^{b′}, —SOR^{b}, —SO₂R^{b}, —SO₂NR^{b′}R^{b′} or C₁₋₆alkyl optionally substituted with one or more substituents R^{c};
R³ represents:
H,
C₁₋₆ alkyl optionally substituted with one or more substituents selected from R^{c} and R^{d}, or
Cy optionally substituted with one or more substituents selected from R^{c}, R^{d} and C₁₋₆alkyl optionally substituted with one or more substituents selected from R^{c} and R^{d};
each R⁴ independently represents H, R^{e}, halogen, —OR^{e′}, —NO₂, —CN, —COR^{e′}, —CO₂R^{e′}, —CONR^{e′}R^{e′}, —NR^{e′}R^{e′}, —NR^{e′}COR^{e′}, —NR^{e′}CONR^{e′}R^{e′}, —NR^{e′}CO₂R^{e}, —NR^{e′}SO₂R^{e}, —SR^{e′}, —SOR^{e}, —SO₂R^{e} or —SO₂NR^{e′}R^{e′};
R⁵ independently represents H, R^{e}, —COR^{e}, —CONR^{e}R^{e}, —SOR^{e} or —SO₂R^{e};
each R^{a} independently represents C₁₋₆alkyl or haloC₁₋₆alkyl;
each R^{b} independently represents C₁₋₆alkyl or Cy, wherein both groups can be optionally substituted with one or more substituents selected from R^{d} and R^{f};
each R^{b′} independently represents H or R^{b};
each R^{c} independently represents halogen, —OR^{g′}, —NO₂, —CN, —COR^{g′}, —CO₂R^{g′}, —CONR^{g′}R^{g′}, —NR^{g′}R^{g′}, —NR^{g′}COR^{g′}, —NR^{g′}CONR^{g′}R^{g′}, —NR^{g′}CO₂R^{g}, —NR^{g′}SO₂R^{g}, —SR^{g′}, —SOR^{g}, —SO₂R^{g} or —SO₂NR^{g′}R^{g′};
R^{d} represents Cy optionally substituted with one or more substituents R^{f};
each R^{e} independently represents C₁₋₆alkyl optionally substituted with one or more substituents selected from R^{c} and Cy*, or R^{e} represents Cy, wherein any of the groups Cy or Cy* can be optionally substituted with one or more substituents selected from R^{c} and R^{g};
each R^{e′} independently represents H or R^{e};
each R^{f} independently represents halogen, R^{h}, —OR^{h′}, —NO₂, —CN, —COR^{h′}, CO₂R^{h′}, —CONR^{h′}R^{h′}, —NR^{h′}R^{h′}, —NR^{h′}COR^{h′}, —NR^{h′}CONR^{h′}R^{h′}, —NR^{h′}CO₂R^{h}, —NR^{h′}SO₂R^{h}, —SR^{h′}, —SOR^{h}, —SO₂R^{h}, or —SO₂NR^{h′}R^{h′};
each R^{g} independently represents R^{d} or C₁₋₆alkyl optionally substituted with one or more substituents selected from R^{d} and R^{f};
each R^{g′} independently represents H or R^{g};
each R^{h} independently represents C₁₋₆alkyl, haloC₁₋₆alkyl or hydroxyC₁₋₆alkyl;
each R^{h′} independently represents H or R^{h}; and
Cy or Cy* in the above definitions represent a partially unsaturated, saturated or aromatic 3- to 7-membered monocyclic or 8- to 12-membered bicyclic carbocyclic ring, which optionally contains from 1 to 4 heteroatoms selected from N, S and O, wherein one or more C, N or S atoms can be optionally oxidized forming CO, N⁺O⁻, SO or SO₂, respectively, and wherein said ring or rings can be bonded to the rest of the molecule through a carbon or a nitrogen atom;
or a salt thereof.

2. A compound according to claim 1 wherein R¹ represents one or more substituents selected from H, R^{a}, halogen and —OR^{a}.

3. A compound according to claim 2 wherein R¹ represents one or two substituents selected from halogen, haloC₁₋₆alkyl and C₁₋₆ alkoxy.

4. A compound according to claim 1 wherein A represents C.

5. A compound according to claim 1 wherein 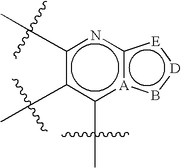
represents a group selected from (a)-(h)

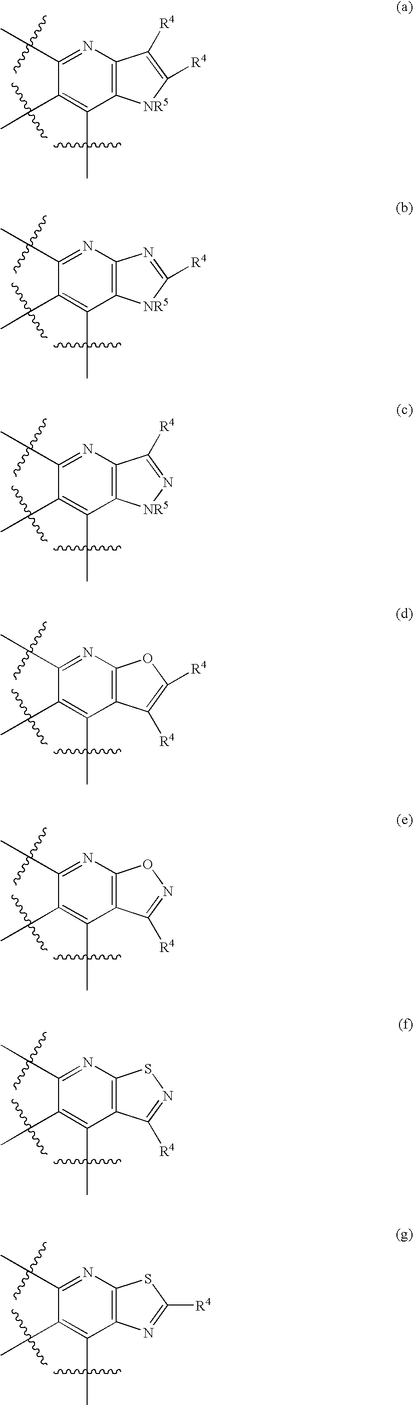

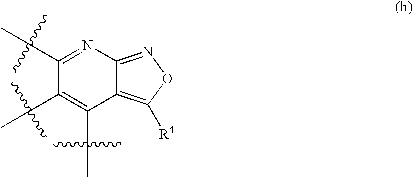

6. A compound according to claim 1 wherein R⁴ independently represents H, R^{e}, —COR^{e′}, —CO₂R^{e′}, —CONR^{e′}R^{e′} or —NR^{e′}R^{e′}.

7. A compound according to claim 1 wherein R⁵ independently represents H or R^{e}.

8. A compound according to claim 7 wherein R⁵ independently represents H or C₁₋₆alkyl.

9. A compound according to claim 1 wherein R² represents one substituent selected from H, halogen, C₁₋₆alkyl, —OR^{b′} and —NR^{b′}R^{b′}.

10. A compound according to claim 1 wherein G represents C and R² represents H.

11. A compound according to claim 1 wherein G represents N, R² represents —NHR^{b} and is placed on the 2-position of the pyrimidine ring, and R^{b} represents C₁₋₆alkyl substituted with one substituent selected from Cy and —OR^{h′}.

12. A compound according to claim 1 wherein R³ represents H, heteroaryl or phenyl, wherein heteroaryl and phenyl can be optionally substituted with one or more substituents selected from R^{c}, R^{d} and C₁₋₆alkyl optionally substituted with one or more substituents selected from R^{c} and R^{d}.

13. A compound according to claim 10 wherein R³ represents phenyl, which can be optionally substituted with one or more halogen.

14. A compound according to claim 11 wherein R³ represents H.

15. A compound according to claim 1 selected from:
methyl 5,7-bis(4-fluorophenyl)-6-(4-pyridyl)thieno[3,2-b]pyridin-3-carboxylate;
methyl 4,6-bis(4-fluorophenyl)-5-(4-pyridyl)furo[2,3-b]pyridine-2-carboxylate;
methyl 5,7-bis(4-fluorophenyl)-1-methyl-6-(4-pyridyl)pyrrolo[3,2-b]pyridine-2-carboxylate;
4,6-bis(4-fluorophenyl)-3-methyl-5-(4-pyridyl)isoxazolo[5,4-b]pyridine;
ethyl 5,7-bis(4-fluorophenyl)-1-methyl-6-(4-pyridyl)imidazo[4,5-b]pyridine-2-carboxylate;
[5,7-bis(4-fluorophenyl)-1-methyl-6-(4-pyridyl)pyrrolo[3,2-b]pyridin-2-yl]methanol;
[5,7-bis(4-fluorophenyl)-1-methyl-6-(4-pyridyl)imidazo[4,5-b]pyridin-2-yl]methanol;
5,7-bis(4-fluorophenyl)-2-methyl-6-(4-pyridyl)pyrazolo[1,5-a]pyrimidine;
2-methyl-5,7-diphenyl-6-(4-pyridyl)pyrazolo[1,5-a]pyrimidine;
5,7-bis(4-fluorophenyl)-1-methyl-6-(4-pyridyl)imidazo[4,5-b]pyridine;
5,7-bis(4-fluorophenyl)-N-(2-hydroxyethyl)-6-(4-pyridyl)thieno[3,2-b]pyridine-3-carboxamide;
5,7-bis(4-fluorophenyl)-1-methyl-6-(4-pyridyl)pyrrolo[3,2-b]pyridine-2-carboxamide;
5,7-bis(4-fluorophenyl)-N-(2-hydroxyethyl)-1-methyl-6-(4-pyridyl)pyrrolo[3,2-b]pyridine-2-carboxamide;
[5,7-bis(4-fluorophenyl)-1-methyl-6-(4-pyridyl)pyrrolo[3,2-b]pyridin-2-yl]morpholin-4-ylmethanone;
3-amino-5,7-bis(4-fluorophenyl)-6-(4-pyridyl)thieno[3,2-b]pyridine;
2-[4,6-bis(4-fluorophenyl)-5-(4-pyridyl)furo[2,3-b]pyridin-2-yl]propan-2-ol;
2-[5,7-bis(4-fluorophenyl)-6-(4-pyridyl)thieno[3,2-b]pyridin-3-yl]propan-2-ol;
2-[5,7-bis(4-fluorophenyl)-1-methyl-6-(4-pyridyl)imidazo[4,5-b]pyridin-2-yl]propan-2-ol;
1-[5,7-bis(4-fluorophenyl)-1-methyl-6-(4-pyridyl)imidazo[4,5-b]pyridin-2-yl]ethanone;
2-[5,7-bis(4-fluorophenyl)-1-methyl-6-(4-pyridyl)pyrrolo[3,2-b]pyridin-2-yl]propan-2-ol;
1-[5,7-bis(4-fluorophenyl)-1-methyl-6-(4-pyridyl)pyrrolo[3,2-b]pyridin-2-yl]ethanone;
[4,6-bis(4-fluorophenyl)-5-(4-pyridyl)furo[2,3-b]pyridin-2-yl]methanol;
4,6-bis-(4-fluoro-phenyl)-5-pyridin-4-yl-furo[2,3-b]pyridine-2-carboxylic acid (2-methoxy-ethyl)-amide;
4,6-bis-(4-fluoro-phenyl)-5-pyridin-4-yl-furo[2,3-b]pyridine-2-carboxylic acid propylamide;
4,6-bis-(4-fluoro-phenyl)-5-pyridin-4-yl-furo[2,3-b]pyridine-2-carboxylic acid (2-morpholin-4-yl-ethyl)-amide;
4,6-bis-(4-fluoro-phenyl)-5-pyridin-4-yl-furo[2,3-b]pyridine-2-carboxylic acid (2-piperidin-1-yl-ethyl)-amide;
4,6-bis-(4-fluoro-phenyl)-5-pyridin-4-yl-furo[2,3-b]pyridine-2-carboxylic acid (2-hydroxy-ethyl)-amide;
5,7-bis-(4-fluoro-phenyl)-1-methyl-6-pyridin-4-yl-1H-pyrrolo[3,2-b]pyridine-2-carboxylic acid (2-methoxy-ethyl)-amide;
5,7-bis-(4-fluoro-phenyl)-1-methyl-6-pyridin-4-yl-1H-pyrrolo[3,2-b]pyridine-2-carboxylic acid propylamide;
5,7-bis-(4-fluoro-phenyl)-1-methyl-6-pyridin-4-yl-1H-pyrrolo[3,2-b]pyridine-2-carboxylic acid (2-morpholin-4-yl-ethyl)-amide;
5,7-bis-(4-fluoro-phenyl)-1-methyl-6-pyridin-4-yl-1H-pyrrolo[3,2-b]pyridine-2-carboxylic acid (2-piperidin-1-yl-ethyl)-amide;
[5,7-bis-(4-fluoro-phenyl)-1-methyl-6-pyridin-4-yl-1H-pyrrolo[3,2-b]pyridin-2-ylmethyl]-(2-methoxy-ethyl)-amine;
[5,7-bis-(4-fluoro-phenyl)-1-methyl-6-pyridin-4-yl-1H-pyrrolo[3,2-b]pyridin-2-ylmethyl]-cyclopropylmethyl-amine;
{[5,7-bis-(4-fluoro-phenyl)-1-methyl-6-pyridin-4-yl-1H-pyrrolo[3,2-b]pyridin-2-ylmethyl]-amino}-acetic acid methyl ester;
{[5,7-bis-(4-fluoro-phenyl)-1-methyl-6-pyridin-4-yl-1H-pyrrolo[3,2-b]pyridin-2-ylmethyl]-N-ethyl-amino}-acetic acid methyl ester;
[5,7-bis-(4-fluoro-phenyl)-1-methyl-6-pyridin-4-yl-1H-pyrrolo[3,2-b]pyridin-2-ylmethyl]-propyl-amine;
5,7-bis-(4-fluoro-phenyl)-1-methyl-6-pyridin-4-yl-1H-pyrrolo[3,2-b]pyridine;
[5,7-bis-(4-fluoro-phenyl)-1-methyl-6-pyridin-4-yl-1H-imidazo[4,5-b]pyridin-2-yl]-morpholin-4-yl-methanone;
5,7-bis-(4-fluoro-phenyl)-6-pyridin-4-yl-1H-pyrazolo[4,3-b]pyridine-3-carboxylic acid methyl ester;
cyclopropylmethyl-{4-[6-(4-fluoro-phenyl)-3-methyl-isoxazolo[5,4-b]pyridin-5-yl]-pyrimidin-2-yl}-amine;
{4-[6-(4-fluoro-phenyl)-3-methyl-isoxazolo[5,4-b]pyridin-5-yl]-pyrimidin-2-yl}-(3-methoxy-propyl)-amine;
(S)-{4-[6-(4-fluoro-phenyl)-3-methyl-isoxazolo[5,4-b]pyridin-5-yl]-pyrimidin-2-yl}-(1-phenyl-ethyl)-amine;
cyclopropylmethyl-{4-[6-(4-fluoro-phenyl)-3-methyl-isothiazolo[5,4-b]pyridin-5-yl]-pyrimidin-2-yl}-amine;
{4-[6-(4-fluoro-phenyl)-3-methyl-isothiazolo[5,4-b]pyridin-5-yl]-pyrimidin-2-yl}-(3-methoxy-propyl)-amine;
(S)-{4-[6-(4-fluoro-phenyl)-3-methyl-isothiazolo[5,4-b]pyridin-5-yl]-pyrimidin-2-yl}-(1-phenyl-ethyl)-amine;
cyclopropylmethyl-{4-[5-(4-methoxy-phenyl)-1H-pyrrolo[3,2-b]pyridin-6-yl]-pyrimidin-2-yl}-amine;
(S)-{4-[5-(4-methoxy-phenyl)-1H-pyrrolo[3,2-b]pyridin-6-yl]-pyrimidin-2-yl}-(1-phenyl-ethyl)-amine;
6-(4-fluoro-phenyl)-4-(2-fluoro-phenyl)-3-methyl-5-pyridin-4-yl-isoxazolo[5,4-b]pyridine;
4,6-bis-(4-fluoro-phenyl)-3-methyl-5-pyridin-4-yl-isothiazolo[5,4-b]pyridine;
4-(2-fluoro-phenyl)-6-(4-fluoro-phenyl)-3-methyl-5-pyridin-4-yl-isothiazolo[5,4-b]pyridine;
3-methyl-5-pyridin-4-yl-6-(3-trifluoromethyl-phenyl)-isoxazolo[3,4-b]pyridine;
cyclopropylmethyl-{4-[5-(4-fluoro-phenyl)-2-methyl-thiazolo[5,4-b]pyridin-6-yl]-pyrimidin-2-yl}-amine;
5,7-bis-(4-fluoro-phenyl)-6-pyridin-4-yl-1H-pyrazolo[4,3-b]pyridine;
5,7-bis-(4-fluoro-phenyl)-6-pyridin-4-yl-1H-pyrazolo[4,3-b]pyridine-3-carboxylic acid (2-hydroxy-ethyl)-amide;
6-(4-fluoro-phenyl)-3-methyl-5-pyridin-4-yl-isoxazolo[3,4-b]pyridine; and
(S)-{4-[5-(4-fluoro-phenyl)-2-methyl-thiazolo[5,4-b]pyridin-6-yl]-pyrimidin-2-yl}-(1-phenyl-ethyl)-amine.

16. A pharmaceutical composition which comprises a compound of formula I according to claim 1 or a pharmaceutically acceptable salt thereof and one or more pharmaceutically acceptable excipients.

17. (canceled)

18. A method for the treatment or prevention of a disease mediated by p38 which comprises administering to a subject in need thereof an effective amount of a compound of formula I according to claim 1 or a pharmaceutically acceptable salt thereof.
